(19) United States
(12) Patent Application Publication
(10) Pub. No.: US 2007/0166230 A1
(43) Pub. Date: Jul. 19, 2007

(54) Ion channel

(76) Inventors: Nicola Brice, Cambridge (GB); Mark Carlton, Cambridge (GB); John Dixon, Cambridge (GB); Isabelle Malinge, Cambridge (GB); Dirk Zahn, Cambridge (GB)

Correspondence Address: FROMMER LAWRENCE & HAUG, 745 FIFTH AVENUE- 10TH FL., NEW YORK, NY, 10151, US

(21) Appl. No.: 11/590,175

(22) Filed: Oct. 30, 2006

Related U.S. Application Data
(63) Continuation-in-part of application No. PCT/GB05/01620, filed on Apr. 28, 2005.

(30) Foreign Application Priority Data
Apr. 28, 2004 (GB) 0409504.8
Oct. 7, 2004 (GB) 0422290.7

(51) Int. Cl. A61K 49/00; G01N 33/567

(52) U.S. Cl. 424/9.2; 435/7.2

(57) ABSTRACT

Provided are Kv9.2 polypeptides comprising the amino acid sequence shown in SEQ ID NO. 3 or SEQ ID NO: 5, and homologues, variants and derivatives thereof. Nucleic acids capable of encoding Kv9.2 polypeptide are also disclosed, in particular, those comprising the nucleic acid sequences shown in SEQ ID No. 1, SEQ ID No.2 or SEQ ID NO: 4. Methods of identifying Kv9.2 agonists and antagonists are also provided.

### CROSS-REFERENCE TO RELATED APPLICATIONS

This application is a continuation-in-part of PCT/GB2005/001620, filed Apr. 28, 2005, published as WO 2005/105838 on Nov. 10, 2005, and claiming priority to GB application nos. 0409504.8 and 0422290.7, filed Apr. 28, 2004 and Oct. 7, 2004, respectively, and to U.S. application Nos. 60/575,626 and 60/617,870, filed May 28, 2004 and Oct. 12, 2004, respectively.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

This invention relates to newly identified nucleic acids, polypeptides encoded by them and to their production and use. More particularly, the nucleic acids and polypeptides of the present invention relate to an ion channel subunit, hereinafter referred to as “Kv9.2”. The invention also relates to inhibiting or activating the action of such nucleic acids and polypeptides.

### BACKGROUND

Ion channels are multi-subunit membrane bound proteins that play a vital role in the functioning of cells. They regulate the passage of a number of ions including sodium, potassium, chloride and calcium across the cellular membrane. As ions carry charge, ion channels are important mediators of fundamental cell electrical properties, including the cell resting potential. Their malfunction and defects have been implicated in many diseases and symptoms including epilepsy, hypertension and cystic fibrosis.

Potassium channels are distributed in the surface membrane of cells and selectively allows potassium ions to pass through it, and it is considered that it takes an important role in controlling membrane potential of cells. Particularly, in nerve and muscle cells, it contributes to the neurotransmission of central and peripheral nerves, pace making of the heart, contraction of muscles and the like by controlling frequency, persistency and the like of action potential. In addition, it has been shown that it is also concerned in the secretion of hormones, adjustment of cell volume, proliferation of cells and the like.

The potassium channel gene family is believed to be the largest and most diverse ion channel family. They have been classified into a number of subfamilies based on the number of transmembrane domains for instance, two, four or six domains. Those with two domains includes GIRK, IRK, CIR and ROMK which have a highly conserved pore domain. Twik-1 and Twik-like channels along with TREK, TASK-1 and 2 and TRAAK have 4 transmembrane domains and are involved in maintaining the steady state potassium ion potentials across the membrane. The Shaker-like and eag type channels have six domains and are the largest sub-family. The Shaker type is a family having markedly high diversity and can be further divided into a number of subfamilies Kv1, Kv2, Kv3, and Kv4. On the other hand, the eag type is constituted by eag, eag-related gene and elk, and it related genes include hyperpolarization activation type potassium channels corresponding to KAT gene cluster and a cation channel which is activated by a cyclic nucleotide.

The first complete nucleotide sequence encoding a Kv channel was reported in 1987 with the cloning of the Shaker channel (Kv1). Low-stringency screening of cDNA libraries with the Shaker cDNA led to isolation of the K+ channel cDNAs Shab (Kv2), Shaw (Kv3) and Shal (Kv4), and that are derived from three distinct genes. The sequences are homologous to Shaker, having ˜40% identity. The Kv1 family, which has >60% homology to Shaker in the core region, is the largest channel family, with at least seven members. In addition to the four mammalian subfamilies relating to Shaker, Shab, Shal, and Shaw, five additional subfamilies (Kv5-9) have also been described. Currently, over 30 Kv channels have been cloned and expressed in heterologous expression systems. These channels often display differences in voltage sensitivity, current kinetics, and steady-state activation and inactivation.

Kv channels exist as tetramers formed by 4 six-transmembrane-spanning-subunits combining to form a functional channel. Not only can identical subunits combine to form a functional channel, but distinct subunits can also combine to form functional heteromeric channels both in vitro and in vivo. These heteromeric channels have unique properties that often represent a blend of the observed properties of the corresponding homomeric channels. Furthermore, several Kv-subunits are nonfunctional when expressed alone. For example, the Kv9.3 subunit, the most recently identified member of the mammalian Kv family, does not form a functional homomeric channel itself but rather functions only in heteromeric complexes where it confers altered voltage sensitivity and kinetics.

Accessory subunits can combine with Kv subunits to add even more diversity to Kv channel function. Currently, four Kv subunit gene families have been described. All are cytoplasmic proteins, ˜40 kDa in mass, with a conserved core sequence and variable NH₂ termini. Kv subunits have been shown to confer functional effects onto subunits, including both fast and slow inactivation, altered voltage sensitivity, and slowed deactivation. Additionally, the subunit may play a role as a cellular redox sensor because it appears to confer O₂ sensitivity on the Kv4.2 channel in heterologous expression systems.

Potassium voltage-gated channel, delayed-rectifier, subfamily S, member-2 (Kv9.2) mRNA had previously been shown to be expressed in pancreatic islets but it was shown not colocalize with insulin, suggesting that it was not involved in the control of insulin secretion (Yan, L., et al. Diabetes 2004. 53. 597-607).

We have now found that the Kv9.2 potassium channel is important in the maintenance of blood sugar. In Kv9.2 knockout animals the blood glucose levels are significantly different (i.e., lower) from that of the wild type animal. The gene, therefore, has control and regulation of the metabolism of sugars and fats.

According to a 1^{st} aspect of the present invention, we provide a transgenic non-human animal having a functionally disrupted endogenous gene, in which the Kv9.2 gene comprises a nucleic acid sequence shown in SEQ ID NO: 1, SEQ ID NO: 2 or SEQ ID NO: 4 or a sequence having at least 70% sequence identity thereto.

Preferably, the transgenic non-human animal has a deletion in a Kv9.2 gene or a portion thereof. Preferably, the transgenic non-human animal displays any one or combination of the following phenotypes: (a) decreased blood glucose levels; (b) increased anxiety, preferably as measured in an Open Field Test and/or a Plus Maze Test; as compared to a wild-type animal.

There is provided, according to a 2^{nd} aspect of the present invention, a transgenic non-human animal in which at least a portion or the whole of the Kv9.2 gene of the animal is replaced with a sequence from the Kv9.2 gene of another animal, preferably another species, more preferably a human.

Preferably, the transgenic non-human animal is a mouse.

Preferably, the transgenic non-human animal comprises a functionally disrupted Kv9.2 gene, preferably a deletion in a Kv9.2 gene, in which the Kv9.2 gene comprises a nucleic acid sequence shown in SEQ ID NO: 4 or a sequence having at least 70% sequence identity thereto.

We provide, according to a 3^{rd} aspect of the present invention, an isolated cell or tissue from a non-human transgenic animal according to the 1^{st} or 2^{nd} aspect of the invention.

As a 4^{th} aspect of the present invention, there is provided a cell having a functionally disrupted endogenous Kv9.2 gene, in which the Kv9.2 gene comprises a nucleic acid sequence shown in SEQ ID NO: 1, SEQ ID NO: 2 or SEQ ID NO: 4, or a sequence having at least 70% sequence identity thereto.

We provide, according to a 5^{th} aspect of the present invention, use of a transgenic non-human animal as described, a cell or tissue as described or a cell as described as a model for anxiety or diabetes.

The present invention, in a 6^{th} aspect, provides use of a transgenic non-human animal as described, a cell or tissue as described or a cell as described as a model for a Kv9.2 associated disease.

In a 7^{th} aspect of the present invention, there is provided use of a transgenic non-human animal as described, a cell or tissue as described or a cell as described in a method of identifying an agonist or antagonist of a Kv9.2 polypeptide comprising an amino acid sequence shown in SEQ ID NO: 3 or SEQ ID NO: 5 or a sequence having at least 70% sequence identity thereto.

According to an 8^{th} aspect of the present invention, we provide a method of identifying an agonist or antagonist of a Kv9.2 polypeptide having an amino acid sequence shown in SEQ ID NO: 3 or SEQ ID NO: 5 or a sequence having at least 70% sequence identity thereto, the method comprising administering a candidate compound to an animal, preferably a wild type animal or a transgenic non-human animal according to the 1^{st} aspect of the invention, and measuring a change in any of the following phenotypes: (a) blood glucose levels; and (b) anxiety, preferably as measured in an Open Field Test and/or a Plus Maze Test.

Preferably, the method identifies an agonist of Kv9.2 polypeptide and comprises identifying a candidate compound capable of causing the animal to display an increase in any of the phenotypes (a)-(b).

Alternatively, or in addition, the method identifies an antagonist of Kv9.2 polypeptide and comprises identifying a candidate compound capable of causing the animal to display any of the phenotypes (a)-(b) or a decrease in such a phenotype.

We provide, according to a 9^{th} aspect of the invention, a method of identifying an agonist or antagonist of a Kv9.2 polypeptide having an amino acid sequence shown in SEQ ID NO: 3 or SEQ ID NO: 5 or a sequence having at least 70% sequence identity thereto, the method comprising exposing a candidate compound to a cell or tissue, preferably a wild type cell or tissue or a cell or tissue as described or a cell as described, and measuring a change in conductance or kinetics of the cell or a cell of the tissue.

Preferably, the method identifies an agonist of Kv9.2 polypeptide and comprises identifying a candidate compound capable of increasing conductance or kinetics of the cell.

Alternatively, or in addition, , the method identifies an antagonist of Kv9.2 polypeptide and comprises identifying a candidate compound capable of decreasing conductance or kinetics of the cell.

There is provided, in accordance with a 10^{th} aspect of the present invention, a method of identifying a compound suitable for the treatment or alleviation of anxiety or diabetes, preferably a Kv9.2 associated disease, the method comprising exposing a Kv9.2 polypeptide comprising an amino acid sequence shown in SEQ ID NO: 3 or SEQ ID NO: 5 or a sequence having at least 70% sequence identity thereto to a candidate compound, and determining whether the candidate compound is an antagonist or antagonist of the Kv9.2 polypeptide.

As an 11^{th} aspect of the invention, we provide use of a Kv9.2 polynucleotide comprising a nucleic acid sequence shown in SEQ ID NO: 1, SEQ ID NO: 2 or SEQ ID NO: 4 or a sequence having at least 70% sequence identity thereto, for the identification of an agonist or antagonist thereof for the treatment of anxiety or diabetes, preferably a Kv9.2 associated disease.

We provide, according to a 12^{th} aspect of the invention, use of a Kv9.2 polypeptide comprising an amino acid sequence shown in SEQ ID NO: 3 or SEQ ID NO: 5 or a sequence having at least 70% sequence identity thereto, for the identification of an agonist or antagonist thereof for the treatment of anxiety or diabetes, preferably a Kv9.2 associated disease.

According to a 13^{th} aspect of the present invention, we provide an antagonist of a Kv9.2 polypeptide having an amino acid sequence shown in SEQ ID NO: 3 or SEQ ID NO: 5 or a sequence having at least 70% sequence identity thereto for use in a method of treatment of anxiety or diabetes, preferably a Kv9.2 associated disease, in an individual.

There is provided, according to a 14^{th} aspect of the present invention, use of an antagonist of a Kv9.2 polypeptide having an amino acid sequence shown in SEQ ID NO: 3 or SEQ ID NO: 5 or a sequence having at least 70% sequence identity thereto for the preparation of a pharmaceutical composition for the treatment of anxiety or diabetes, preferably a Kv9.2 associated disease, in an individual.

We provide, according to a 15^{th} aspect of the present invention, a method of treating an individual suffering from anxiety or diabetes, preferably suffering from a Kv9.2 associated disease, the method comprising administering an antagonist of Kv9.2 to the individual.

According to a 16^{th} aspect of the present invention, we provide a method of diagnosis of anxiety or diabetes, preferably a Kv9.2 associated disease, in an individual, the method comprising detecting a change in expression, level or activity of Kv9.2 in the individual or a cell or tissue thereof.

Preferably, the Kv9.2 associated disease is selected from the group consisting of: Type I and Type II diabetes, hyperinsulinaemia, hyperinsulinism, insulin resistance, complications of diabetes including diabetes associated vascular disease, diabetes associated renal disease and diabetes associated neuropathy, and the treatment of hypoglycaemia, hyperlipoidemia (be they HDL, LDL or VLDL), dyslipoidemia, whether primary in origins or secondary to diabetes, hyper/hypothyroidism, acromegaly, liver failure, renal failure, pancreatic tumours, pancreatitis and alcohol induced hypoglycaemia.

Alternatively, or in addition, the Kv9.2 associated disease is selected from the group consisting of: social anxiety, post traumatic stress disorder, phobias, social phobia, special phobias, panic disorder, obsessive compulsive disorder, acute stress, disorder, separation anxiety disorder, generalised anxiety disorder, major depression, dysthymia, bipolar disorder, seasonal affective disorder, post natal depression, manic depression, bipolar depression, anxiety, anxiety disorders, anxiety-related behaviour and generalized anxiety disorder, agoraphobia, acute stress disorder, and those panic disorders list in DSM-IV, and depression.

According to a 17^{th} aspect of the present invention, we provide a Kv9.2 polypeptide comprising the amino acid sequence shown in SEQ ID NO. 3 or SEQ ID NO: 5, or a homologue, variant or derivative thereof having at least 70% sequence identity thereto.

We provide, according to an 18^{th} aspect of the present invention, a nucleic acid encoding a polypeptide according to the 18^{th} aspect of the invention.

Preferably, the nucleic acid comprises a sequence shown in SEQ ID No. 1, SEQ ID No.2 or SEQ ID NO: 4, or a homologue, variant or derivative thereof having at least 70% sequence identity thereto.

### BRIEF DESCRIPTION OF THE DRAWINGS

FIG. 1 is a diagram showing a knockout vector for creating Kv9.2 deficient mice.

FIG. 2 shows Kv9.2 gene-expression results from the human RT-PCR screen.

FIG. 3 is a graph showing the results of the analysis of blood glucose levels (mmol/L).

FIGS. 4A-4E show the nucleotide sequence of the knockout plasmid vector (SEQ ID NO: 19).

FIGS. 5A-5C show the results of the Open Field Test FIG. 5A shows total distance traveled in peripheral (left) and central (right) areas (filled column knockout animals); FIG. 5B shows time moving in peripheral (left) and central (right) areas; (filled columns knockout animals) and FIG. 5C shows number of entries into filed zone (filled column knockout animals).

FIG. 6 is a graph of the analysis of the results of the Plus Maze test showing time spent in the closed arms and the open arms of the maze (filled columns knockout animals and hatched columns wildtype animals).

### SEQUENCE LISTINGS

SEQ ID NO: 1 shows the cDNA sequence of human Kv9.2. SEQ ID NO: 2 shows an open reading frame derived from SEQ ID NO: 1. SEQ ID NO: 3 shows the amino acid sequence of human Kv9.2. SEQ ID NO: 4 shows the open reading frame of a cDNA for Mouse Kv9.2. SEQ ID NO: 5 shows the amino acid sequence of Mouse Kv9.2. SEQ ID NOs. 6-18 show the genotyping primers used to construct the knockout plasmid. SEQ ID NOs: 19 shows the knockout plasmid vector sequence.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855, Lars-Inge Larsson “Immunocytochemistry: Theory and Practice”, CRC Press Inc., Boca Raton, Fla., 1988, ISBN 0-8493-6078-1, John D. Pound (ed); “Immunochemical Protocols, vol 80”, in the series: “Methods in Molecular Biology”, Humana Press, Totowa, N.J., 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3, and The Merck Manual of Diagnosis and Therapy (17th Edition, Beers, M. H., and Berkow, R, Eds, ISBN: 0911910107, John Wiley & Sons). Each of these general texts is herein incorporated by reference. Each of these general texts is herein incorporated by reference.

### DETAILED DESCRIPTION

Kv9.2 Subunit

Our invention relates in general to the use of an ion channel and a subunit thereof, in particular, to Kv9.2 subunit of the voltage gated potassium channel, as well as homologues, variants or derivatives thereof, in the treatment, relief or diagnosis of Kv9.2 associated diseases, including diabetes or anxiety. This and other embodiments of the invention will be described in further detail below.

Expression Profile of Kv9.2 Subunit

As shown in the Examples, polymerase chain reaction (PCR) amplification of Kv9.2 cDNA detects expression of Kv9.2 to varying abundance in a number of organs including prostate, liver, reproductive organs, muscle and brain.

Using Kv9.2 cDNA of SEQ ID NO: 1 to search the human EST data sources by BLASTN, identities are found in cDNA libraries. This indicates that Kv9.2 is expressed in normal or abnormal tissues such as,

| | |
|---|---|
| U69192 | Human Infant brain |
| AA776703 | Human Testis |
| A1681499 | Human Lung |
| AW292826 | Human ovary |

Accordingly, the Kv9.2 polypeptides, nucleic acids, probes, antibodies, expression vectors and ligands are useful for detection, diagnosis, treatment and other assays for diseases associated with over-, under- and abnormal expression of Kv9.2 subunit in these and other tissues.

Kv9.2 Subunit Associated Diseases

According to the methods and compositions described here, Kv9.2 subunit is useful for treating and diagnosing a range of diseases. These diseases are referred to for convenience as Kv9.2 associated diseases.

We demonstrate here that human Kv9.2 maps to Homo sapiens chromosome 8q22. Accordingly, in a specific embodiment, Kv9.2 subunit may be used to treat or diagnose a disease which maps to this locus, chromosomal band, region, arm or the same chromosome. Known diseases which have been determined as being linked to the same locus, chromosomal band, region, arm or chromosome as the chromosomal location of Kv9.2 subunit (i.e., Homo sapiens chromosome 8q22) include Renal tubular acidosis-osteopetrosis syndrome, Dihydropyrimidinuria, Cohen syndrome, and Klippel-Feil syndrome with laryngeal malformation. However, to date no specific diseases have been associated with the channel where channel has been found to be up or down regulated.

Knockout mice deficient in Kv9.2 display a range of phenotypes, as demonstrated in the Examples.

In particular, Example 5 demonstrates that blood glucose levels in Kv9.2 knockout mice are significantly higher than corresponding wild type mice. A deficit of Kv9.2 activity is therefore correlated with a decrease in blood sugar levels.

We therefore disclose a method of lowering blood sugar levels in an individual, preferably for the treatment of diabetes, the method comprising decreasing the level or activity of Kv9.2 in that individual. As noted elsewhere, this can be achieved by down-regulating the expression of Kv9.2, or by use of antagonists to Kv9.2.

Particularly, the modulation of blood glucose by such means may be used for the treatment of diseases including, but not limited to, Type I and Type II diabetes, hyperinsulinaemia, hyperinsulinism, insulin resistance, complications of diabetes including diabetes associated vascular disease, diabetes associated renal disease and diabetes associated neuropathy, and the treatment of hypoglycaemia. Further, it may also be used for the treatment of hyperlipoidemia (be they HDL, LDL or VLDL), and dyslipoidemia, whether primary in origins or secondary to diabetes, hyper/hypothyroidism, acromegaly, liver failure, renal failure, pancreatic tumours, pancreatitis and alcohol induced hypoglycaemia. Kv9.2 knockout mice may therefore furthermore be used as models for any of these diseases.

Furthermore, Example 6 describes an Open Field test, in which Kv9.2 knockout mice are shown to be more anxious than their wild type counterparts. A deficit of Kv9.2 activity is therefore correlated with a increase in stress.

We therefore disclose a method of lowering stress or anxiety or both in an individual, the method comprising increasing the level or activity of Kv9.2 in that individual. As noted elsewhere, this can be achieved by up-regulating the expression of Kv9.2, or by use of agonists to Kv9.2.

Kv9.2 and modulators of Kv9.2 activity, including in particular antagonists of Kv9.2, may be used to treat or alleviate diseases or syndromes in which stress and anxiety feature. Such diseases include social anxiety, post traumatic stress disorder, phobias, social phobia, special phobias, panic disorder, obsessive compulsive disorder, acute stress, disorder, separation anxiety disorder, generalised anxiety disorder, major depression, dysthymia, bipolar disorder, seasonal affective disorder, post natal depression, manic depression, bipolar depression. Kv9.2 knockout mice may therefore furthermore be used as models for any of these diseases.

In a preferred embodiment, the Kv9.2 associated disease comprises a disease in which stress or anxiety is a symptom. In a highly preferred embodiment, the Kv9.2 disease comprises the above list of anxiety and stress related diseases.

In addition, the gene has also been found to have an effect on neurotic disorders including anxiety, anxiety disorders, anxiety-related behaviour and generalized anxiety disorder, panic disorder, agoraphobia, social phobia, obsessive-compulsive disorder, posttraumatic stress disorder, acute stress disorder, and those panic disorders list in DSM-IV, and depression.

As noted above, Kv9.2 subunit may be used to diagnose and/or treat any of these specific diseases using any of the methods and compositions described here.

In particular, we specifically envisage the use of nucleic acids, vectors comprising Kv9.2 nucleic acids, polypeptides, including homologues, variants or derivatives thereof, pharmaceutical compositions, host cells, and transgenic animals comprising Kv9.2 nucleic acids and/or polypeptides, for the treatment or diagnosis of the specific diseases listed above. Furthermore, we envisage the use of compounds capable of interacting with or binding to Kv9.2, preferably antagonists of a Kv9.2 hetero or homomeric ion channel, preferably a compound capable modulating the kinetics or reducing the conductance of the channel, antibodies against Kv9.2 subunit, as well as methods of making or identifying these, in diagnosis or treatment of the specific diseases mentioned above. In particular, we include the use of any of these compounds, compositions, molecules, etc., in the production of vaccines for treatment or prevention of the specific diseases. We also disclose diagnostic kits for the detection of the specific diseases in an individual.

Methods of linkage mapping to identify such or further specific diseases treatable or diagnosable by use of Kv9.2 subunit are known in the art, and are also described elsewhere in this document.

Anxiety and Stress

Anxiety and stress, as well as disorders in which these are manifested, including Kv9.2 associated diseases, are well known in the art. A summary description follows:

Anxiety and stress are also referred to as feeling uptight, tension, jitters, and apprehension. Stress can come from any situation or thought that makes an individual feel frustrated, angry, or anxious. What is stressful to one person is not necessarily stressful to another.

Anxiety is a feeling of apprehension or fear. The source of this uneasiness is not always known or recognized, which can add to the distress the individual feels.

Stress is a normal part of life. In small quantities, stress is may be beneficial—it can motivate an individual and him to be more productive. However, too much stress, or a strong response to stress, is harmful. It can set the individual up for general poor health as well as specific physical or psychological illnesses like infection, heart disease, or depression. Persistent and unrelenting stress often leads to anxiety and unhealthy behaviours like overeating and abuse of alcohol or drugs.

Emotional states like grief or depression and health conditions like an overactive thyroid, low blood sugar, or heart attack can also cause stress.

Anxiety is often accompanied by physical symptoms, including: twitching or trembling, muscle tension, headaches, sweating, dry mouth, difficulty swallowing, abdominal pain (this may be the only symptom of stress, especially in a child)

Sometimes other symptoms accompany anxiety: dizziness, rapid or irregular heart rate, rapid breathing, diarrhoea or frequent need to urinate, fatigue, irritability, including loss of temper, sleeping difficulties and nightmares, decreased concentration and sexual problems.

Kv9.2 and its modulators may be used to treat or alleviate any of these symptoms.

Anxiety disorders are a group of psychiatric conditions that involve excessive anxiety. They include generalized anxiety disorder, specific phobias, obsessive-compulsive disorder, and social phobia. See also Kv9.2 associated diseases set out above.

Certain drugs, both recreational and medicinal, can lead to symptoms of anxiety due to either side effects or withdrawal from the drug. Such drugs include caffeine, alcohol, nicotine, cold remedies, decongestants, bronchodilators for asthma, tricyclic antidepressants, cocaine, amphetamines, diet pills, ADHD medications, and thyroid medications. We disclose the use of Kv9.2 and its modulators in combination with such drugs to alleviate their stress and/or anxiety inducing effects.

A poor diet can also contribute to stress or anxiety—for example, low levels of vitamin B12. Performance anxiety is related to specific situations, like taking a test or making a presentation in public. Post-traumatic stress disorder (PTSD) is a stress disorder that develops after a traumatic event like war, physical or sexual assault, or a natural disaster.

In very rare cases, a tumor of the adrenal gland (pheochromocytoma) may be the cause of anxiety. This happens because of an overproduction of hormones responsible for the feelings and symptoms of anxiety.

(Adapted from Medline Plus, found on the website maintained by the National Library of Medicine.)

Identities and Similarities and to Kv9.2 Subunit

Kv9.2 is structurally related to other proteins of the ion channel family, as shown by the results of sequencing the amplified cDNA products encoding human Kv9.2. The cDNA sequence of SEQ ID NO: 1 contains an open reading flame (SEQ ID NO: 2, nucleotide numbers 41 to 3352) encoding a polypeptide of 1104 amino acids shown in SEQ ID NO: 3. Human Kv9.2 is found to map to Homo sapiens chromosome 8q22.

Analysis of the Kv9.2 polypeptide (SEQ ID NO: 3) using the HMM structural prediction software of pfam (available at the pfam website maintained by the Sanger Institute) confirms that Kv9.2 peptide is an ion channel subunit.

The mouse homologue of the human Kv9.2 subunit has been cloned, and its nucleic acid sequence and amino acid sequence are shown as SEQ ID NO: 4 and SEQ ID NO: 5 respectively. The mouse Kv9.2 subunit cDNA of SEQ ID NO: 4 shows a high degree of identity with the human Kv9.2 subunit (SEQ ID NO: 2) sequence, while the amino acid sequence (SEQ ID NO: 5) of mouse Kv9.2 subunit shows a high degree of identity and similarity with human Kv9.2 subunit (SEQ ID NO: 3). Human and mouse Kv9.2 ion channel subunit are therefore members of a large family of ion channels.

Kv9.2 Subunit Polypeptides

As used here, the term “Kv9.2 subunit polypeptide” is intended to refer to a polypeptide comprising the amino acid sequence shown in SEQ ID No. 3 or SEQ ID NO: 5, or a homologue, variant or derivative thereof. Preferably, the polypeptide comprises or is a homologue, variant or derivative of the sequence shown in SEQ ID NO: 3.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-inking, cyclization, disulfide bond formation, demethylation, formation of covalent cross-inks, formation of cystine, formation of pyroglutamate, formulation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983, Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

The terms “variant”, “homologue”, “derivative” or “fragment” as used in this document include any substitution of, variation of, modification of, replacement of, deletion of or addition of one (or more) amino acid from or to a sequence. Unless the context admits otherwise, references to “Kv9.2”, “Kv9.2 subunit” and “Kv9.2 ion channel” include references to such variants, homologues, derivatives and fragments of Kv9.2.

Preferably, as applied to Kv9.2, the resultant amino acid sequence has ion channel activity when expressed to form homomeric channels or in combination with other Kv family members to form heteromeric channels. Preferably, the resultant nucleic acid has the same activity (or potential for activity when combined with other channels as indicated) as the Kv9.2 ion channel subunit shown as SEQ ID NO: 3 or SEQ ID NO: 5.

In particular, the term “homologue” covers identity with respect to structure and/or function providing the resultant amino acid sequence has ion channel activity, preferably Kv9.2 ion channel activity, when combined with other channels as indicated. With respect to sequence identity (i.e. similarity), preferably there is at least 70%, more preferably at least 75%, more preferably at least 85%, even more preferably at least 90% sequence identity. More preferably there is at least 95%, more preferably at least 98%, sequence identity. These terms also encompass polypeptides derived from amino acids which are allelic variations of the Kv9.2 subunit nucleic acid sequence.

Where reference is made to the “channel activity” or “biological activity” of an ion channel such as Kv9.2 containing ion channel, these terms are intended to refer to the metabolic or physiological function of the Kv9.2 containing ion channel, including similar activities or improved activities or these activities with decreased undesirable side effects. Also included are antigenic and immunogenic activities of the Kv9.2 containing ion channel. Examples of ion channel activity, and methods of assaying and quantifying these activities, are known in the art, and are described in detail elsewhere in this document.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring substance. As used herein “substitution” results from the replacement of one or more nucleotides or amino acids by different nucleotides or amino acids, respectively.

Kv9.2 polypeptides described here may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine, and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Kv9.2 polypeptides may further comprise heterologous amino acid sequences, typically at the N-terminus or C-terminus, preferably the N-terminus. Heterologous sequences may include sequences that affect intra or extracellular protein targeting (such as leader sequences). Heterologous sequences may also include sequences that increase the immunogenicity of the polypeptide and/or which facilitate identification, extraction and/or purification of the polypeptides. Another heterologous sequence that is particularly preferred is a polyamino acid sequence such as polyhistidine which is preferably N-terminal. A polyhistidine sequence of at least 10 amino acids, preferably at least 17 amino acids but fewer than 50 amino acids is especially preferred.

The Kv9.2 polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Kv9.2 polypeptides are advantageously made by recombinant means, using known techniques. However they may also be made by synthetic means using techniques well known to skilled persons such as solid phase synthesis. Such polypeptides may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. Preferably the fusion protein will not hinder the function of the protein of interest sequence.

Kv9.2 polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the Kv9.2 ion channel protein may be mixed with carriers or diluents which will not interfere with the intended purpose of the protein and still be regarded as substantially isolated. A polypeptide as described here may also be in a substantially purified form, in which case it will generally comprise the protein in a preparation in which more than 90%, for example, 95%, 98% or 99% of the protein in the preparation is a Kv9.2 polypeptide.

We further describe peptides comprising a portion of a Kv9.2 polypeptide. Thus, fragments of Kv9.2 subunit and its homologues, variants or derivatives are included. The peptides may be between 2 and 200 amino acids, preferably between 4 and 40 amino acids in length. The peptide may be derived from a Kv9.2 polypeptide as disclosed here, for example by digestion with a suitable enzyme, such as trypsin. Alternatively the peptide, fragment, etc. may be made by recombinant means, or synthesised synthetically.

The term “peptide” includes the various synthetic peptide variations known in the art, such as a retroinverso D peptides. The peptide may be an antigenic determinant and/or a T-cell epitope. The peptide may be immunogenic in vivo. Preferably the peptide is capable of inducing neutralising antibodies in vivo.

By aligning Kv9.2 subunit sequences from different species, it is possible to determine which regions of the amino acid sequence are conserved between different species (“homologous regions”), and which regions vary between the different species (“heterologous regions”).

The Kv9.2 polypeptides may therefore comprise a sequence which corresponds to at least part of a homologous region. A homologous region shows a high degree of homology between at least two species. For example, the homologous region may show at least 70%, preferably at least 80%, more preferably at least 90%, even more preferably at least 95% identity at the amino acid level using the tests described above. Peptides which comprise a sequence which corresponds to a homologous region may be used in therapeutic strategies as explained in further detail below. Alternatively, the Kv9.2 subunit peptide may comprise a sequence which corresponds to at least part of a heterologous region. A heterologous region shows a low degree of homology between at least two species.

Kv9.2 Polynucleotides and Nucleic Acids

We describe Kv9.2 polynucleotides, Kv9.2 nucleotides and Kv9.2 nucleic acids, methods of production, uses of these, etc., as described in further detail elsewhere in this document.

The terms “Kv9.2 polynucleotide”, “Kv9.2 nucleotide” and “Kv9.2 nucleic acid” may be used interchangeably, and are intended to refer to a polynucleotide/nucleic acid comprising a nucleic acid sequence as shown in SEQ ID NO: 1, SEQ ID NO: 2 or SEQ ID NO: 4, or a homologue, variant or derivative thereof. Preferably, the polynucleotide/nucleic acid comprises or is a homologue, variant or derivative of the nucleic acid sequence SEQ ID NO: 1 or SEQ ID NO: 2, most preferably, SEQ ID NO: 2.

These terms are also intended to include a nucleic acid sequence capable of encoding a polypeptides and/or a peptide, i.e., a Kv9.2 polypeptide. Thus, Kv9.2 polynucleotides and nucleic acids comprise a nucleotide sequence capable of encoding a polypeptide comprising the amino acid sequence shown in SEQ ID NO: 3 or SEQ ID NO: 5, or a homologue, variant or derivative thereof. Preferably, the Kv9.2 polynucleotides and nucleic acids comprise a nucleotide sequence capable of encoding a polypeptide comprising the amino acid sequence shown in SEQ ID NO: 3, or a homologue, variant or derivative thereof.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

Preferably, the term “nucleotide sequence” means DNA.

The terms “variant”, “homologue”, “derivative” or “fragment” as used here include any substitution of, variation of, modification of, replacement of, deletion of or addition of one (or more) nucleic acids from or to the sequence of a Kv9.2 nucleotide sequence. Unless the context admits otherwise, references to “Kv9.2”, “Kv9.2 subunit” and “Kv9.2 ion channel” include references to such variants, homologues, derivatives and fragments of Kv9.2.

Preferably, the resultant nucleotide sequence encodes a polypeptide having Kv9.2 subunit activity, preferably having at least the same activity of the Kv9.2 subunit shown as SEQ ID NO: 3 or SEQ ID NO: 5. Preferably, the term “homologue” is intended to cover identity with respect to structure and/or function. Preferably, this is such that the resultant nucleotide sequence encodes a polypeptide which has ion channel activity when expressed to form homomeric channels or in combination with other Kv family members to form heteromeric channels. With respect to sequence identity (i.e. similarity), preferably there is at least 70%, more preferably at least 75%, more preferably at least 85%, more preferably at least 90% sequence identity. More preferably there is at least 95%, more preferably at least 98%, sequence identity. These terms also encompass allelic variations of the sequences.

Calculation of Sequence Homology

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Other computer program methods to determine identify and similarity between the two sequences include but are not limited to the GCG program package (Devereux et al. 1984 Nucleic Acids Research 12: 387) and FASTA (Atschul et al. 1990 J Molec Biol 403-410).

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (Ausubel et al., 1999 ibid, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail at the National Center for Biotechnology Information website, which is incorporated herein by reference. Search parameters can be defined and can be advantageously set over the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see National Center for Biotechnology Information website) with a few enhancements. The BLAST programs are tailored for sequence similarity searching, for example to identify homologues to a query sequence. For a discussion of basic issues in similarity searching of sequence databases, see Altschul et al. (1994) Nature Genetics 6:119-129.

The five BLAST programs available at the National Center for Biotechnology Information website perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see National Center for Biotechnology Information website). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at the National Center for Biotechnology Information website. In some embodiments, no gap penalties are used when determining sequence identity.

Hybridisation

This document also encompasses nucleotide sequences that are capable of hybridising to the sequences presented herein, or any fragment or derivative thereof, or to the complement of any of the above.

Hybridization means a “process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Nucleotide sequences of capable of selectively hybridising to the nucleotide sequences presented herein, or to their complement, will be generally at least 70%, preferably at least 75%, more preferably at least 85 or 90% and even more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences will comprise regions homologous to SEQ ID NO: 1, 2 or 4, preferably at least 70%, 80% or 90% and more preferably at least 95% homologous to one of the sequences.

The term “selectively hybridizable” means that the nucleotide sequence used as a probe is used under conditions where a target nucleotide sequence is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Also included within the scope of this document are nucleotide sequences that are capable of hybridizing to the nucleotide sequences presented herein under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm, intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred embodiment, we describe nucleotide sequences that can hybridise to one or more of the Kv9.2 subunit nucleotide sequences under stringent conditions (e.g. 65° C. and 0.1×SSC {1×SSC=0.15 M NaCl, 0.015 M Na₃ Citrate pH 7.0}). Where the nucleotide sequence is double-stranded, both strands of the duplex, either individually or in combination, are encompassed. Where the nucleotide sequence is single-stranded, it is to be understood that the complementary sequence of that nucleotide sequence is also included within the scope of this document.

We further describe nucleotide sequences that are capable of hybridising to the sequences that are complementary to the sequences presented herein, or any fragment or derivative thereof. Likewise, nucleotide sequences that are complementary to sequences that are capable of hybridising to the sequence described here are included. These types of nucleotide sequences are examples of variant nucleotide sequences. In this respect, the term “variant” encompasses sequences that are complementary to sequences that are capable of hydridising to the nucleotide sequences presented herein. Preferably, however, the term “variant” encompasses sequences that are complementary to sequences that are capable of hydridising under stringent conditions (e.g. 65° C. and 0.1×SSC {1×SSC=0.15 M NaCl, 0.015 Na₃ citrate pH 7.0}) to the nucleotide sequences presented herein.

Cloning of Kv9.2 Subunit and Homologues

We further describe nucleotide sequences that are complementary to the sequences presented here, or any fragment or derivative thereof. If the sequence is complementary to a fragment thereof then that sequence can be used as a probe to identify and clone similar subunit sequences in other organisms etc.

The disclosure of this document thus enables the cloning of Kv9.2, its homologues and other structurally or functionally related genes from human and other species such as mouse, pig, sheep, etc. to be accomplished. Polynucleotides which are identical or sufficiently identical to a nucleotide sequence contained in SEQ ID NO: 1, SEQ ID NO: 2, SEQ ID NO: 4 or a fragment thereof, may be used as hybridization probes for cDNA and genomic DNA, to isolate partial or full-length cDNAs and genomic clones encoding Kv9.2 subunit from appropriate libraries. Such probes may also be used to isolate cDNA and genomic clones of other genes (including genes encoding homologues and orthologues from species other than human) that have sequence similarity, preferably high sequence similarity, to the Kv9.2 gene. Hybridization screening, cloning and sequencing techniques are known to those of skill in the art and are described in, for example, Sambrook et al. (supra).

Typically nucleotide sequences suitable for use as probes are 70% identical, preferably 80% identical, more preferably 90% identical, even more preferably 95% identical to that of the referent. The probes generally will comprise at least 15 nucleotides. Preferably, such probes will have at least 30 nucleotides and may have at least 50 nucleotides. Particularly preferred probes will range between 150 and 500 nucleotides, more particularly about 300 nucleotides.

In one embodiment, to obtain a polynucleotide encoding a Kv9.2 polypeptide, including homologues and orthologues from species other than human, comprises the steps of screening an appropriate library under stringent hybridization conditions with a labelled probe having the SEQ ID NO: 1, SEQ ID NO: 2, SEQ ID NO: 4 or a fragment thereof and isolating partial or full-length cDNA and genomic clones containing said polynucleotide sequence. Such hybridization techniques are well known to those of skill in the art. Stringent hybridization conditions are as defined above or alternatively conditions under overnight incubation at 42 degrees C. in a solution comprising: 50% formamide, 5×SSC (150 mM NaCl, 15 mM trisodium citrate), 50 mM sodium phosphate (pH7.6), 5 ×Denhardt's solution, 10% dextran sulphate, and 20 microgram/ml denatured, sheared salmon sperm DNA, followed by washing the filters in 0.1×SSC at about 65 degrees C.

Functional Assay for Kv9.2 Containing Ion Channels

The cloned putative Kv9.2 ion channel polynucleotides may be verified by sequence analysis or functional assays. In particular, the conductance of Xenopus oocytes tranfected as described may be detected as a means of gauging and quantifying Kv9.2 activity, useful for screening assays described below. Such a Xenopus oocyte electrophysiology assay is referred to for convenience as a “Functional Assay of Kv9.2 (Electrophysiology)”.

The putative Kv9.2 ion channel subunit or homologue may be assayed for activity in a “Functional Assay of Kv9.2 (Electrophysiology)” as follows. Capped RNA transcripts from linearized plasmid templates encoding the Kv9.2 cDNAs are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. RNA encoding other Kv subunits e.g. Kv2.1, Kv4.2 may also be injected to form heteromeric channels. Two electrode voltage clamps are used to measure the currents from individual Xenopus oocytes in response to agonist exposure. Recordings of the current are made in standard medium consisting of (in mM) NaCl 115, KCl 2.5, CaCl₂ 1.8, NaOH-HEPES 10, pH7.2 at room temperature. The Xenopus system may also be used to screen known ligands and tissue/cell extracts for activating ligands, as described in further detail below.

Alternative functional assays include patch clamp electrophysiology, Rb flux, fluorescence resonance energy transfer (FRET) analysis and FLIPR analysis, including the use of voltage sensitive dyes to investigate the membrane voltage of the cell. A FLIPR assay is described in Whiteaker et al. J Biomol Screen. 2001 October; 6(5):305-1, while a FRET based assay is described in Falconer et al. J Biomol Screen. 2002 October; 7(5):460-5.

Specifically, we disclose an assay which detects Rb flux, as well as screens which detect change in Rb flux to identify agonists and antagonists of Kv9.2. Methods for measuring radiolabelled Rb flux are outlined in Rezazadeh et al. J Biomol Screen. 2004 October; 9(7):588-97 and a non-radiolabelled Rb flux assay in Assay Drug Dev Technol. 2004 October; 2(5):525-34. Preferably, % Rb efflux is measured in the assay.

Such a functional assay is referred to in this document as a “Functional Assay for Kv9.2 (Rb flux)”.

Specifically, we disclose a method in which antagonists of Kv9.2 lower % Rb efflux of a suitably transfected cell. Preferably, the % Rb efflux is lowered by 10%, 20%, 30%, 40%, 50%, 60%, 70% or more in the presence of an antagonist of Kv9.2.

We further disclose a method in which agonists of Kv9.2 increase the % Rb efflux of a suitably transfected cell. Preferably, the % Rb efflux is increased by 10%, 20%, 30%, 40%, 50%, 60%, 70% or more in the presence of an agonist of Kv9.2.

The kinetic analysis of efflux Rb+ release from the cells can be expressed as the percentage remaining ® using the following equation
R=[Rb_{lysate}/(Rbₛᵤₚₑᵣₙ+Rb_{lysate})]×100

Depolarisation and agonist-stimulated Rb+ efflux (Rₛ) at different time points can be determined according to
Rₛ=(1−[(R_{tot-R}−R_{basal})/−R_{basal}])×100

The Kv9.2 polypeptide may further be assayed for its kinetics, which include the activation, deactivation and inactivation. The activation time is the time taken for a full current to be established across a Kv9.2 containing channel under standard conditions, which the deactivation time is the time taken for a full current to zero under standard conditions. Where reference is made to modulation, increase or decrease of Kv9.2 kinetics, this should be taken to refer preferably to modulation, increase or decrease of Kv9.2 activation time, or Kv9.2 deactivation time, or both.

In preferred embodiments, the activation time constant is used as a measure of activation time, and the deactivation time constant is used as a measure of deactivation time. A typical activation time constant for Kv9.2 containing channels is 21 ms. A typical potential for half-inactivation V_{1/2 inact} is −33 mV, and the V_{1/2 inact} may be assayed as a further or alternative kinetic parameter of inactivation.

Modulators, such as openers, agonists, blockers and antagonists of Kv9.2 containing channels are capable of changing, i.e., increasing or decreasing, the kinetics of the Kv9.2 containing channel, preferably any one or more of the activation time, the inactivation time, deactivation time, deactivation kinetics, potential for half-inactivation, etc.

In particular, agonists and openers are molecules which are capable of decreasing the activation time and/or deactivation time (preferably the activation time and/or deactivation time constant), preferably by 10%, 20%, 30%, 40%, 50%, 60%, 70%, 80%, 90% or more, i.e., by decreasing the activation time to 20 ms, 18 ms, 16ms, or 15 ms or less, for example.

Similarly, antagonists or blockers of Kv9.2 are capable of increasing the activation time and/or deactivation time, preferably by 10%, 20%, 30%, 40%, 50%, 60%, 70%, 80%, 90% or more, i.e., by increasing the activation time to 22 ms, 25 ms or 27 ms or more, for example.

The kinetics and specifically the activation time may be preferably measured using the “Functional Assay of Kv9.2 (Electrophysiology)”, taking a time course of current and establishing the time taken for full current to be established. Similarly, the inactivation time is measured using the “Functional Assay of Kv9.2 (Electrophysiology)”, taking a time course of current and establishing the time taken for the full current to fall to zero.

Alternatively, the deactivation kinetics, which are a measure of the time the channel takes to deactivate after a repolarising pulse (e.g. to −40 mV) after a prepulse (e.g. +50 mV for 500 ms), may be assayed. A typical value for the deactivation kinetics of Kv9.2 containing channels is 44 ms.

Expression Assays for Kv9.2

In order to design useful therapeutics for treating Kv9.2 associated diseases, it is useful to determine the expression profile of Kv9.2 (whether wild-type or a particular mutant). Thus, methods known in the art may be used to determine the organs, tissues and cell types (as well as the developmental stages) in which Kv9.2 is expressed. For example, traditional or “electronic” Northerns may be conducted. Reverse-transcriptase PCR (RT-PCR) may also be employed to assay expression of the Kv9.2 gene or mutant. More sensitive methods for determining the expression profile of Kv9.2 include RNAse protection assays, as known in the art.

Northern analysis is a laboratory technique used to detect the presence of a transcript of a gene and involves the hybridization of a labelled nucleotide sequence to a membrane on which RNAs from a particular cell type or tissue have been bound. (Sambrook, supra, ch. 7 and Ausubel, F. M. et al. supra, ch. 4 and 16.) Analogous computer techniques (“electronic Northerns”) applying BLAST may be used to search for identical or related molecules in nucleotide databases such as GenBank or the LIFESEQ database (Incyte Pharmaceuticals). This type of analysis has advantages in that they may be faster than multiple membrane-based hybridizations. In addition, the sensitivity of the computer search can be modified to determine whether any particular match is categorized as exact or homologous.

The polynucleotides and polypeptides described here may be employed as research reagents and materials for discovery of treatments and diagnostics to animal and human disease, as explained in further detail elsewhere in this document.

Expression of Kv9.2 Polypeptides

We further disclose a process for producing a Kv9.2 polypeptide. The method comprises in general culturing a host cell comprising a nucleic acid encoding Kv9.2 polypeptide with or without other Kv family members, or a homologue, variant, or derivative thereof, under suitable conditions (i.e., conditions in which the Kv9.2 polypeptide is expressed).

In order to express a biologically active Kv9.2 containing ion channels, the nucleotide sequences encoding Kv9.2 subunit or homologues, variants, or derivatives thereof are inserted into appropriate expression vector, i.e., a vector which contains the necessary elements for the transcription and translation of the inserted coding sequence.

Methods which are well known to those skilled in the art are used to construct expression vectors containing sequences encoding Kv9.2 subunit and appropriate transcriptional and translational control elements. These methods include iii vitro recombinant DNA techniques, synthetic techniques, and in vivo genetic recombination. Such techniques are described in Sambrook, J. et al. (1989; Molecular Cloning, A Laboratory Manual, ch. 4, 8, and 16-17, Cold Spring Harbor Press, Plainview, N.Y.) and Ausubel, F. M. et al. (1995 and periodic supplements; Current Protocols in Molecular Biology, ch. 9, 13, and 16, John Wiley & Sons, New York, N.Y.).

A variety of expression vector/host systems may be utilized to contain and express sequences encoding Kv9.2 subunit. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. Such a use is not limited by the host cell employed.

The “control elements” or “regulatory sequences” are those non-translated regions of the vector (i.e., enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (GIBCO/BRL), and the like, may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding Kv9.2 subunit, vectors based on SV40 or EBV may be used with an appropriate selectable marker.

In bacterial systems, a number of expression vectors may be selected depending upon the use intended for Kv9.2 subunit. For example, when large quantities of Kv9.2 subunit are needed for the induction of antibodies, vectors which direct high level expression of fusion proteins that are readily purified may be used. Such vectors include, but are not limited to, multifunctional E. coli cloning and expression vectors such as BLUESCRIPT (Stratagene), in which the sequence encoding Kv9.2 subunit may be ligated into the vector in frame with sequences for the amino-terminal Met and the subsequent 7 residues of β-galactosidase so that a hybrid protein is produced, pIN vectors (Van Heeke, G. and S. M. Schuster (1989) J. Biol. Chem. 264:5503-5509), and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. For reviews, see Ausubel (supra) and Grant et al. (1987; Methods Enzymol. 153:516-544).

In cases where plant expression vectors are used, the expression of sequences encoding Kv9.2 subunit may be driven by any of a number of promoters. For example, viral promoters such as the 35S and 19S promoters of CaMV may be used alone or in combination with the omega leader sequence from TMV. (Takamatsu, N. (1987) EMBO J. 6:307-311.) Alternatively, plant promoters such as the small subunit of RUBISCO or heat shock promoters may be used. (Coruzzi, G. et al. (1984) EMBO J. 3:1671-1680; Broglie, R. et al. (1984) Science 224:838-843, and Winter, J. et al. (1991) Results Probl. Cell Differ. 17:85-105.). These constructs can be introduced into plant cells by direct DNA transformation or pathogen-mediated transfection. Such techniques are described in a number of generally available reviews. (See, for example, Hobbs, S. or Murry, L. E. in McGraw Hill Yearbook of Science and Technology (1992) McGraw Hill, New York, N.Y.; pp. 191-196.).

An insect system may also be used to express Kv9.2 subunit. For example, in one such system, Autographa californica nuclear polyhedrosis virus (AcNPV) is used as a vector to express foreign genes in Spodoptera frugiperda cells or in Trichoplusia larvae. The sequences encoding Kv9.2 subunit may be cloned into a non-essential region of the virus, such as the polyhedrin gene, and placed under control of the polyhedrin promoter. Successful insertion of Kv9.2 subunit will render the polyhedrin gene inactive and produce recombinant virus lacking coat protein. The recombinant viruses may then be used to infect, for example, S. frugiperda cells or Trichoplusia larvae in which Kv9.2 containing ion channel may be expressed. (Engelhard, E. K. et al. (1994) Proc. Nat. Acad. Sci. 91:3224-3227.)

In mammalian host cells, a number of viral-based expression systems may be utilized. In cases where an adenovirus is used as an expression vector, sequences encoding Kv9.2 subunit may be ligated into an adenovirus transcription/translation complex consisting of the late promoter and tripartite leader sequence. Insertion in a non-essential E1 or E3 region of the viral genome may be used to obtain a viable virus which is capable of expressing Kv9.2 subunit in infected host cells. (Logan, J. and T. Shenk (1984) Proc. Natl. Acad. Sci. 81:3655-3659.) In addition, transcription enhancers, such as the Rous sarcoma virus (RSV) enhancer, may be used to increase expression in mammalian host cells.

Thus, for example, Kv9.2 containing channels are expressed in either human embryonic kidney 293 (HEK293) cells or adherent CHO cells. To maximize channel expression, typically all 5′ and 3′ untranslated regions (UTRs) are removed from the receptor cDNA prior to insertion into a pCDN or pCDNA3 vector. The cells are transfected with individual channel cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual channels, about 24 clones are typically selected and analyzed by Northern blot analysis. Channel mRNAs are generally detectable in about 50% of the G418-resistant clones analyzed.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding Kv9.2 subunit. Such signals include the ATG initiation codon and adjacent sequences. In cases where sequences encoding Kv9.2 subunit and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used, such as those described in the literature. (Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC, Bethesda, Md.) and may be chosen to ensure the correct modification and processing of the foreign protein.

For long term, high yield production of recombinant proteins, stable expression is preferred. For example, cell lines capable of stably expressing Kv9.2 homo or heteromeric ion channels can be transformed using expression vectors which may contain viral origins of replication and/or endogenous expression elements and a selectable marker gene on the same or on a separate vector. Following the introduction of the vectors, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes (Wigler, M. et al. (1977) Cell 11:223-32) and adenine phosphoribosyltransferase genes (Lowy, I. et al. (1980) Cell 22:817-23), which can be employed in tk⁻ or apr⁻ cells, respectively. Also, antimetabolite, antibiotic, or herbicide resistance can be used as the basis for selection. For example, dhfr confers resistance to methotrexate (Wigler, M. et al. (1980) Proc. Natl. Acad. Sci. 77:3567-70); npt confers resistance to the aminoglycosides neomycin and G-418 (Colbere-Garapin, F. et al. (1981) J. Mol. Biol. 150:1-14); and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine. (Hartman, S. C. and R. C. Mulligan (1988) Proc. Natl. Acad. Sci. 85:8047-51.) Recently, the use of visible markers has gained popularity with such markers as anthocyanins, β-glucuronidase and its substrate GUS, and luciferase and its substrate luciferin. These markers can be used not only to identify transformants, but also to quantify the amount of transient or stable protein expression attributable to a specific vector system. (Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding Kv9.2 subunit is inserted within a marker gene sequence, transformed cells containing sequences encoding Kv9.2 subunit can be identified by the absence of marker gene function. Alternatively, a marker gene can be placed in tandem with a sequence encoding Kv9.2 subunit under the control of a single promoter. Expression of the marker gene in response to induction or selection usually indicates expression of the tandem gene as well.

Alternatively, host cells which contain the nucleic acid sequence encoding Kv9.2 subunit and express Kv9.2 subunit may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein sequences.

The presence of polynucleotide sequences encoding Kv9.2 subunit can be detected by DNA-DNA or DNA-RNA hybridization or amplification using probes or fragments or fragments of polynucleotides encoding Kv9.2 subunit. Nucleic acid amplification based assays involve the use of oligonucleotides or oligomers based on the sequences encoding Kv9.2 subunit to detect transformants containing DNA or RNA encoding Kv9.2 subunit.

A variety of protocols for detecting and measuring the expression of Kv9.2 subunit, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on Kv9.2 subunit is preferred, but a competitive binding assay may be employed. These and other assays are well described in the art, for example, in Hampton, R. et al. (1990; Serological Methods, a Laboratory Manual, Section IV, APS Press, St Paul, Minn.) and in Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding Kv9.2 subunit include oligolabeling, nick translation, end-labeling, or PCR amplification using a labeled nucleotide. Alternatively, the sequences encoding Kv9.2 subunit, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding Kv9.2 subunit may be cultured under conditions suitable for the expression and recovery of the protein from cell culture. The protein produced by a transformed cell may be located in the cell membrane, secreted or contained intracellularly depending on the sequence and/or the vector used. As will be understood by those of skill in the art, expression vectors containing polynucleotides which encode Kv9.2 subunit may be designed to contain signal sequences which direct secretion of Kv9.2 subunit through a prokalyotic or eukaryotic cell membrane. Other constructions may be used to join sequences encoding Kv9.2 subunit to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the Kv9.2 subunit encoding sequence may be used to facilitate purification. One such expression vector provides for expression of a fusion protein containing Kv9.2 subunit and a nucleic acid encoding 6 histidine residues preceding a thioredoxin or an enterokinase cleavage site. The histidine residues facilitate purification on immobilized metal ion affinity chromatography (IMIAC, described in Porath, J. et al. (1992) Prot. Exp. Purif. 3: 263-281), while the enterokinase cleavage site provides a means for purifying Kv9.2 subunit from the fusion protein. A discussion of vectors which contain fusion proteins is provided in Kroll, D. J. et al. (1993, DNA Cell Biol. 12:441-453).

Fragments of Kv9.2 subunit may be produced not only by recombinant production, but also by direct peptide synthesis using solid-phase techniques. (Merrifield J. (1963) J. Am. Chem. Soc. 85:2149-2154.) Protein synthesis may be performed by manual techniques or by automation. Automated synthesis may be achieved, for example, using the Applied Biosystems 431A peptide synthesizer (Perkin Elmer). Various fragments of Kv9.2 subunit may be synthesized separately and then combined to produce the full length molecule.

Biosensors

The Kv9.2 polypeptides, nucleic acids, probes, antibodies, expression vectors and ligands are useful as (and for the production of) biosensors.

According to Aizawa (1988), Anal. Chem. Symp. 17: 683, a biosensor is defined as being a unique combination of a target for molecular recognition, for example a selective layer with immobilized antibodies or channel such as Kv9.2, and a transducer for transmitting the values measured. One group of such biosensors will detect the change which is caused in the optical properties of a surface layer due to the interaction of the channel with the surrounding medium. Among such techniques may be mentioned especially ellipso-metry and surface plasmon resonance. Biosensors incorporating Kv9.2 may be used to detect the presence or level of Kv9.2 ligands. The construction of such biosensors is well known in the art.

Thus, cell lines expressing Kv9.2 may be used as reporter systems for detection of ligands such as ATP via receptor-promoted formation of [3H]inositol phosphates or other second messengers (Watt et al., 1998, J Biol Chem May 29; 273(22):14053-8). Receptor-ligand biosensors are also described in Hoffman et al., 2000, Proc Natl Acad Sci USA October 10; 97(21): 11215-20. Optical and other biosensors comprising Kv9.2 may also be used to detect the level or presence of interaction with G-proteins and other proteins, as described by, for example, Figler et al., 1997, Biochemistry December 23; 36(51): 16288-99 and Sarrio et al., 2000, Mol Cell Biol 2000 July; 20(14):5164-74). Sensor units for biosensors are described in, for example, U.S. Pat. No. 5,492,840.

Screening Assays

The Kv9.2 polypeptide, including homologues, variants, and derivatives, whether natural or recombinant, may be employed in a screening process for compounds which bind the Kv9.2 subunit, or Kv9.2 containing ion channel and which activate (agonists) or inhibit activation of (antagonists or blockers) of Kv9.2. Thus, such polypeptides may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2):Chapter 5 (1991).

Kv9.2 ion channel polypeptides are responsible for many biological functions, including many pathologies. Accordingly, it is desirous to find compounds and drugs which stimulate Kv9.2 containing ion channels on the one hand and which can inhibit the function of Kv9.2 ion channels on the other hand. In general, agonists and antagonists are employed for therapeutic and prophylactic purposes for such conditions as anxiety, stress, depression or diabetes.

Rational design of candidate compounds likely to be able to interact with Kv9.2 ion channel proteins may be based upon structural studies of the molecular shapes of a polypeptide. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general producing appropriate cells which express the Kv9.2 ion channel polypeptide on the surface thereof. Such cells include cells from animals, yeast, Drosophila or E. coli. Cells expressing Kv9.2 (or cell membrane containing the expressed protein) are then contacted with a test compound to observe binding, or stimulation or inhibition of a functional response. For example, Xenopus oocytes may be injected with Kv9.2 mRNA or polypeptide, and currents induced by exposure to test compounds measured by use of voltage clamps measured, as described in further detail elsewhere.

Instead of testing each candidate compound individually with the Kv9.2 subunit or Kv9.2 containing ion channel, a library or bank of candidate ligands may advantageously be produced and screened. Thus, for example, a bank of over 200 putative ligands has been assembled for screening. The bank comprises: transmitters, hormones and chemokines known to act via an ion channel; naturally occurring compounds which may be putative agonists for an ion channel, non-mammalian, biologically active peptides for which a mammalian counterpart has not yet been identified; and compounds not found in nature, but which activate ion channels with unknown natural ligands.

This bank may be used to screen the Kv9.2 subunit or Kv9.2 containing ion channel for known ligands, using both functional (e.g., Rb flux assay, FRET assay, FLIPR assay, whole cell electrophysiology, oocyte electrophysiology, etc., see elsewhere) as well as binding assays as described in further detail elsewhere. However, a large number of mammalian channels exist for which there remains, as yet, no cognate activating ligand (agonist) or deactivating ligand (antagonist). Thus, active ligands for these receptors may not be included within the ligands banks as identified to date. Accordingly, Kv9.2 may also be functionally screened (using oocyte electrophysiology, etc., functional screens) against tissue extracts to identify natural ligands. Extracts that produce positive functional responses can be sequentially subfractionated, with the fractions being assayed as described here, until an activating ligand is isolated and identified.

Another method involves screening for ion channel inhibitors by determining inhibition or stimulation of Kv9.2 containing ion channels. Such a method involves transfecting a eukaryotic cell with the Kv9.2 subunits either alone to form a homomeric channel or with other Kv channel subunits to form a heteromeric channel to express the ion channel on the cell surface. The cell is then exposed to potential antagonists in the presence of the Kv9.2 containing ion channel. The cell can be tested using whole cell electrophysiology to determine the changes in the conductance or kinetics of the current.

Another method for detecting agonists or antagonists of Kv9.2 is the yeast based technology as described in U.S. Pat. No. 5,482,835, incorporated by reference herein.

In a preferred embodiment, the screen employs detection of a change in conductance to screen for agonists and antagonists of Kv9.2. Specifically, we disclose a method in which antagonists of Kv9.2 lower the conductance of a suitably transfected cell. Preferably, the conductance is lowered by 10%, 20%, 30%, 40%, 50%, 60%, 70% or more in the presence of an antagonist of Kv9.2. Preferably, the conductance is lowered by 1 pS, 2 pS, 3 pS, 4 pS, 5 pS, 10 pS, 15 pS, 25 pS, 35 pS, 45 pS, 60 pS, 70 pS or more in the presence of an antagonist of Kv9.2.

We further disclose a method in which agonists of Kv9.2 increase the conductance of a suitably transfected cell. Preferably, the conductance is increased by 10%, 20%, 30%, 40%, 50%, 60%, 70% or more in the presence of an agonist of Kv9.2. Preferably, the conductance is increased by 1 pS, 2 pS, 3 pS, 4 pS, 5 pS, 10 pS, 15 pS, 25 pS, 35 pS, 45 pS, 60 pS, 70 pS or more in the presence of an agonist of Kv9.2.

In a further preferred embodiment, the screen employs detection of a change in radiolabelled Rb flux, preferably % Rb efflux, to screen for agonists and antagonists of Kv9.2. Preferably, the screen employs a function assay as set out above under “Functional Assay of Kv9.2 (Rb flux)”.

Specifically, we disclose a method in which antagonists of Kv9.2 lower % Rb efflux of a suitably transfected cell. Preferably, the % Rb efflux is lowered by 10%, 20%, 30%, 40%, 50%, 60%, 70% or more in the presence of an antagonist of Kv9.2.

We further disclose a method in which agonists of Kv9.2 increase the % Rb efflux of a suitably transfected cell. Preferably, the % Rb efflux is increased by 10%, 20%, 30%, 40%, 50%, 60%, 70% or more in the presence of an agonist of Kv9.2.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a Kv9.2 polypeptide or peptide are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses. Competitive assays are particularly useful, where the cells expressing the library of compounds are contacted or incubated with a labelled antibody known to bind to a Kv9.2 polypeptide, such as ¹²⁵I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a Kv9.2 polypeptide, and is described in detail in International Patent application No. WO 84/03564 (Commonwealth Serum Labs.), published on Sep. 13, 1984. First, large numbers of different small peptide test compounds are synthesized on a solid substrate, e.g., plastic pins or some other appropriate surface; see Fodor et al. (1991). Then all the pins are reacted with solubilized Kv9.2 polypeptide and washed. The next step involves detecting bound polypeptide. Compounds which interact specifically with the polypeptide will thus be identified.

Ligand binding assays provide a direct method for ascertaining pharmacology and are adaptable to a high throughput format. The purified ligand may be radiolabeled to high specific activity (50-2000 Ci/mmol) for binding studies. A determination is then made that the process of radiolabeling does not diminish the activity of the ligand towards its target. Assay conditions for buffers, ions, pH and other modulators such as nucleotides are optimized to establish a workable signal to noise ratio for both membrane and whole cell receptor or ion channel sources. For these assays, specific binding is defined as total associated radioactivity minus the radioactivity measured in the presence of an excess of unlabeled competing ligand. Where possible, more than one competing ligand is used to define residual nonspecific binding.

The assays may simply test binding of a candidate compound wherein adherence to the cells bearing the receptor or ion channel is detected by means of a label directly or indirectly associated with the candidate compound or in an assay involving competition with a labeled competitor. Further, these assays may test whether the candidate compound results in a signal generated by activation of the target, using detection systems appropriate to the cells bearing the target at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing a Kv9.2 polypeptide to form a mixture, measuring Kv9.2 containing ion channel activity in the mixture, and comparing the Kv9.2 ion channel activity of the mixture to a standard.

The Kv9.2 subunit cDNA, protein and antibodies to the protein may also be used to configure assays for detecting the effect of added compounds on the production of Kv9.2 subunit mRNA and protein in cells. For example, an ELISA may be constructed for measuring secreted or cell associated levels of Kv9.2 subunit protein using monoclonal and polyclonal antibodies by standard methods known in the art, and this can be used to discover agents which may inhibit or enhance the production of Kv9.2 subunit (also called antagonist or agonist, respectively) from suitably manipulated cells or tissues. Standard methods for conducting screening assays are well understood in the art.

Examples of potential Kv9.2 ion channel antagonists and blockers include antibodies or, in some cases, nucleotides and their analogues, including purines and purine analogues, oligonucleotides or proteins which are closely related to the ligand of the Kv9.2 containing ion channel, e.g., a fragment of the ligand, or small molecules which bind to the ion channel but do not elicit a response, so that the activity of the channel is prevented.

We there therefore also provide a compound capable of binding specifically to a Kv9.2 polypeptide and/or peptide.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Preferably the compound is an antibody.

The materials necessary for such screening to be conducted may be packaged into a screening kit. Such a screening kit is useful for identifying agonists, antagonists, ligands, receptors, substrates, enzymes, etc. for Kv9.2 polypeptides or compounds which decrease or enhance the production of Kv9.2 ion channel polypeptides. The screening kit comprises: (a) a Kv9.2 polypeptide; (b) a recombinant cell expressing a Kv9.2 polypeptide; (c) a cell membrane expressing a Kv9.2 polypeptide; or (d) antibody to a Kv9.2 polypeptide. The screening kit may optionally comprise instructions for use.

Transgenic Animals

We further disclose transgenic animals capable of expressing natural or recombinant Kv9.2 subunit and/or Kv9.2 containing ion channel, or a homologue, variant or derivative, at normal, elevated or reduced levels compared to the normal expression level. Preferably, such a transgenic animal is a non-human mammal, such as a pig, a sheep or a rodent. Most preferably the transgenic animal is a mouse or a rat.

We disclose transgenic animals in which all or a portion of the native Kv9.2 gene is replaced by Kv9.2 sequences from another organism. Preferably this organism is another species, most preferably a human. In highly preferred embodiments, we disclose a mouse which has substantially its entire Kv9.2 gene replaced with a human Kv9.2 gene. Such transgenic animals, as well as animals which are wild type for Kv9.2, may be used for screening agonists and/or antagonists of Kv9.2.

For example, such assays may involve exposing the wild type or transgenic animal, or a portion thereof, preferably a cell, tissue or organ of the transgenic animal, to a candidate substance, and assaying for a Kv9.2 associated phenotype such as anxiety or decreased blood sugar level. Cell-based screens employing cells derived from the relevant animal and assaying for effects on conductance or kinetics may also be conducted.

We further disclose transgenic animals comprising functionally disrupted Kv9.2 gene, in which any one or more of the functions of Kv9.2 as disclosed in this document is partially or totally abolished. Included are transgenic animals (“Kv9.2 knockout”s) which do not express functional Kv9.2 containing ion channel as a result of one or more loss of function mutations, including a deletion, of the Kv9.2 gene.

Also included are partial loss-of-function mutants, e.g., an incomplete knockout, which may for example have deletions in selected portions of the Kv9.2 gene. Such animals may be generated by selectively replacing or deleting relevant portions of the Kv9.2 sequence, for example, functionally important protein domains.

Such complete or partial loss of function mutants are useful as models for Kv9.2 related diseases, particularly anxiety and diabetes. An animal displaying partial-loss-of-function may be exposed to a candidate substance to identify substances which enhance the phenotype, that is to say, to increase (in the case of Kv9.2) the hypoalgesia or reduction of stress level phenotype observed. Other parameters such as reduction in conductance or kinetics may also be detected using the methods identified elsewhere in this document.

Partial and complete knockouts may also be used to identify selective agonists and/or antagonists of Kv9.2. For example, an agonist and/or antagonist may be administered to a wild type and a Kv9.2 deficient animal (knockout). A selective agonist or antagonist of Kv9.2 will be seen to have an effect on the wild type animal but not in the Kv9.2 deficient animal. In detail, a specific assay is designed to evaluate a potential drug (a candidate ligand or compound) to determine if it produces a physiological response in the absence of Kv9.2 containing ion channel. This may be accomplished by administering the drug to a transgenic animal as discussed above, and then assaying the animal for a particular response. Analogous cell-based methods employing cells derived from the relevant animal and assaying for effects on conductance or kinetics may also be conducted. Such animals may also be used to test for efficacy of drugs identified by the screens described in this document.

In another embodiment, a transgenic animal having a partial loss-of-function phenotype is employed for screening. In such an embodiment, the screen may involve assaying for partial or complete restoration or reversion to the wild type phenotype. Cell-based screens employing cells derived from the relevant animal and assaying for effects on conductance or kinetics may also be conducted. A candidate compound which is found to be capable of such can be regarded as a Kv9.2 agonist or analogue. Such agonists may be used for example to increase blood sugar levels, or to decrease stress or anxiety levels in an individual.

In preferred embodiments, the transgenic Kv9.2 animals, particularly Kv9.2 knockouts (complete loss of function), display the phenotypes set out in the Examples, preferably as measured by the tests set out therein. Thus, the Kv9.2 animals, particularly Kv9.2 knockouts, preferably display any one or more of the following: decreased blood sugar levels, increased anxiety.

In highly preferred embodiments, the transgenic Kv9.2 animals, particularly Kv9.2 knockouts, display at least 10%, preferably at least 20%, more preferably at least30%, 40%, 50%, 60%, 70%, 80%, 90%, or 100% higher or lower (as the case may be) of the measured parameter as compared to the corresponding wild-type mice. Thus, for example, when tested in an Open Field analysis as set out in the Examples, Kv9.2 deficient mice preferably have a statistically increased immobility or decreased ambulation time and/or an increased peripheral permanence time, when compared to wild type mice. A decrease in the total distance moved is also seen.

It will be evident that the phenotypes now disclosed for Kv9.2 deficient transgenic animals may be usefully employed in a screen using wild type animals, to detect compounds which cause similar effects to loss-of-function of Kv9.2. In other words, a wild type animal may be exposed to a candidate compound, and a change in a relevant Kv9.2 phenotype observed, such as anxiety levels, blood glucose levels, etc., to identify modulators of Kv9.2 function, particularly antagonists. Cellular phenotypes such as reduction in conductance or change or reduction in kinetics may also be detected using the methods identified elsewhere in this document.

A compound identified by such a screen could be used as an antagonist of Kv9.2, e.g., as an analgesic or a stress reliever, particularly for the treatment or relief of a Kv9.2 associated disease.

The screens described above may involve observation of any suitable parameter, such as a behavioural, physiological or biochemical response. Preferred responses include physiological responses and may comprise one or more of the following: changes to disease resistance, altered inflammatory responses, altered tumour susceptability: a change in blood pressure, neovascularization, a change in eating behavior, a change in body weight, a change in bone density; a change in body temperature, insulin secretion, gonadotropin secretion, nasal and bronchial secretion; vasoconstriction, loss of memory, anxiety; changed anxiety state, hyporeflexia or hypereflexia, or stress responses.

Biochemical parameters may also be employed, such as a change in conductance or kinetics. Preferably, the conductance is measured using the “Functional Assay for Kv9.2 (Electrophysiology)” and the kinetics (activation and/or deactivation time, preferably the activation time and/or deactivation time constant) is measured as described in that section. This is particularly useful in cell-based screens.

In preferred embodiments, the conductance of a cell (for example a wild type or partial loss-of-function cell) exposed to a Kv9.2 agonist is increased by at least 10%, preferably at least 20%, more preferably at least +30%, more preferably at least 40%, more preferably at least 50%, more preferably at least 60%, more preferably at least 70%, more preferably at least 80%, more preferably at least 90%. In preferred embodiments, this is measured using the “Functional Assay for Kv9.2 (Electrophysiology)” described elsewhere in this document.

In preferred embodiments, the kinetics, e.g., the activation time and/or the deactivation time of a cell (for example a wild type or partial loss-of-function cell), preferably the activation time and/or deactivation time constant exposed to a Kv9.2 agonist is increased by at least 10%, preferably at least 20%, more preferably at least +30%, more preferably at least 40%, more preferably at least 50%, more preferably at least 60%, more preferably at least 70%, more preferably at least 80%, more preferably at least 90%. In preferred embodiments, this is measured using the “Functional Assay for Kv9.2 (Electrophysiology)” described elsewhere in this document.

In preferred embodiments, the blood glucose levels of a wild type or Kv9.2 partial knockout animal exposed to a Kv9.2 agonist is increased by at least 10%, preferably at least 20%, more preferably at least +30%, more preferably at least 40%, more preferably at least 50%, more preferably at least 60%, more preferably at least 70%, more preferably at least 80%, more preferably at least 90%.

In preferred embodiments, antagonists of Kv9.2 are such that wild type or partial loss-of-function animals exposed to such antagonists exhibit at least partial identity of phenotype, to at least a partial degree, as Kv9.2 partial or complete loss-of-function mutants. That is to say, preferred antagonists are those which cause increase in anxiety, or decrease in blood sugar levels, or reduction in conductance, or change or reduction in kinetics, or any combination of the above. Preferably, the relevant phenotype is expressed to the same degree as a Kv9.2 knock-out animal.

In preferred embodiments, the conductance of a wild type or partial loss-of-function cell exposed to a Kv9.2 antagonist is within +80%, preferably within +70%, more preferably within +60%, more preferably within +50%, more preferably within +40%, more preferably within +30%, more preferably within +20%, more preferably within +10%, more preferably within +5%, of the conductance of a Kv9.2 deficient cell. In preferred embodiments, this is measured using the “Functional Assay for Kv9.2 (Electrophysiology)” described elsewhere in this document.

In preferred embodiments, the kinetics, i.e., activation time and/or deactivation time, preferably the activation time and/or deactivation time constant, of a wild type or Kv9.2 partial loss-of-function cell exposed to a Kv9.2 antagonist is within +80%, preferably within +70%, more preferably within +60%, more preferably within +50%, more preferably within +40%, more preferably within +30%, more preferably within +20%, more preferably within +10%, more preferably within +5%, of the kinetics (or relevant time) of a Kv9.2 deficient cell. In preferred embodiments, this is measured using the “Functional Assay for Kv9.2 (Electrophysiology)” described elsewhere in this document.

In preferred embodiments, the blood glucose levels of a wild type or partial Kv9.2 knockout animal exposed to a Kv9.2 antagonist is within +80%, preferably within +70%, more preferably within +60%, more preferably within +50%, more preferably within +40%, more preferably within +30%, more preferably within +20%, more preferably within +10%, more preferably within +5%, of the blood glucose levels of a Kv9.2 deficient transgenic animal.

Tissues derived from the Kv9.2 knockout animals may be used in binding assays to determine whether the potential drug (a candidate ligand or compound) binds to the Kv9.2. Such assays can be conducted by obtaining a first ion channel preparation from the transgenic animal engineered to be deficient in Kv9.2 containing ion channel production and a second ion channel preparation from a source known to bind any identified Kv9.2 ligands or compounds. In general, the first and second ion channel preparations will be similar in all respects except for the source from which they are obtained. For example, if brain tissue from a transgenic animal (such as described above and below) is used in an assay, comparable brain tissue from a normal (wild type) animal is used as the source of the second ion channel preparation. Each of the ion channel preparations is incubated with a ligand known to bind to Kv9.2 containing ion channels, both alone and in the presence of the candidate ligand or compound. Preferably, the candidate ligand or compound will be examined at several different concentrations.

The extent to which binding by the known ligand is displaced by the test compound is determined for both the first and second ion channel preparations. Tissues derived from transgenic animals may be used in assays directly or the tissues may be processed to isolate membranes or membrane proteins, which are themselves used in the assays. A preferred transgenic animal is the mouse. The ligand may be labeled using any means compatible with binding assays. This would include, without limitation, radioactive, enzymatic, fluorescent or chemiluminescent labeling (as well as other labelling techniques as described in further detail above).

Furthermore, antagonists of Kv9.2 or Kv9.2 containing ion channels may be identified by administering candidate compounds, etc., to wild type animals expressing functional Kv9.2, and animals identified which exhibit any of the phenotypic characteristics associated with reduced or abolished expression of Kv9.2 function.

Detailed methods for generating non-human transgenic animal are described in further detail below. Transgenic gene constructs can be introduced into the germ line of an animal to make a transgenic mammal. For example, one or several copies of the construct may be incorporated into the genome of a mammalian embryo by standard transgenic techniques.

In an exemplary embodiment, the transgenic non-human animals are produced by introducing transgenes into the germline of the non-human animal. Embryonal target cells at various developmental stages can be used to introduce transgenes. Different methods are used depending on the stage of development of the embryonal target cell. The specific line(s) of any animal are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the Kv9.2 transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity may also be conducted. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. If one or more copies of the exogenous cloned construct remains stably integrated into the genome of such transgenic embryos, it is possible to establish permanent transgenic mammal lines carrying the transgenically added construct.

The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this document, a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a pair of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. There will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the methods described here will include exogenous genetic material. As set out above, the exogenous genetic material will, in certain embodiments, be a DNA sequence which results in the production of a Kv9.2 subunit or Kv9.2 containing ion channel. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156, Bradley et al. (1984) Nature 309:255-258, Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

We also provide non-human transgenic animals, where the transgenic animal is characterized by having an altered Kv9.2 gene, preferably as described above, as models for Kv9.2 subunit or Kv9.2 containing ion channel function. Alterations to the gene include deletions or other loss of function mutations, introduction of an exogenous gene having a nucleotide sequence with targeted or random mutations, introduction of an exogenous gene from another species, or a combination thereof. The transgenic animals may be either homozygous or heterozygous for the alteration. The animals and cells derived therefrom are useful for screening biologically active agents that may modulate Kv9.2 subunit or Kv9.2 containing ion channel function. The screening methods are of particular use for determining the specificity and action of potential therapies for the modulation of blood glucose, the treatment of diseases including, Type I and Type II diabetes, hyperinsulinaemia, hyperinsulinism, insulin resistance, complications of diabetes including diabetes associated vascular disease, diabetes associated renal disease and diabetes associated neuropathy, and the treatment of hypoglycaemia. Also, the treatment of hyperlipoidemia (be they HDL, LDL or VLDL), and dyslipoidemia, whether primary in origins or secondary to diabetes, hyper/hypothyroidism, acromegaly, liver failure, renal failure, pancreatic tumours, pancreatitis and alcohol induced hypoglycaemia. Also for treatment of neurotic disorders including anxiety, anxiety disorders, anxiety-related behaviour and generalized anxiety disorder, panic disorder, agoraphobia, social phobia, obsessive-compulsive disorder, posttraumatic stress disorder, acute stress disorder, and those panic disorders list in DSM-IV, and depression.

The animals are useful as a model to investigate the role of Kv9.2 subunit or Kv9.2 containing ion channels in normal tissues and organs such as the brain, heart, spleen and liver and the effect on their function.

Another aspect pertains to a transgenic nonhuman animal having a functionally disrupted endogenous Kv9.2 gene but which also carries in its genome, and expresses, a transgene encoding a heterologous Kv9.2 protein (i.e., a Kv9.2 from another species). Preferably, the animal is a mouse and the heterologous Kv9.2 is a human Kv9.2. An animal, or cell lines derived from such an animal, which has been reconstituted with human Kv9.2, can be used to identify agents that inhibit human Kv9.2 in vivo and in vitro. For example, a stimulus that induces signalling through human Kv9.2 can be administered to the animal, or cell line, in the presence and absence of an agent to be tested and the response in the animal, or cell line, can be measured. An agent that inhibits human Kv9.2 in vivo or in vitro can be identified based upon a decreased response in the presence of the agent compared to the response in the absence of the agent.

We also provide for a Kv9.2 deficient transgenic non-human animal (a “Kv9.2 subunit knock-out”). Such an animal is one which expresses lowered or no Kv9.2 subunit or Kv9.2 containing ion channel activity, preferably as a result of an endogenous Kv9.2 subunit genomic sequence being disrupted or deleted. Preferably, such an animal expresses no Kv9.2 subunit or Kv9.2 containing ion channel activity. More preferably, the animal expresses no activity of the Kv9.2 containing ion channel shown as SEQ ID NO: 3 or SEQ ID NO: 5. Kv9.2 ion channel knock-outs may be generated by various means known in the art, as described in further detail below.

The present disclosure also pertains to a nucleic acid construct for functionally disrupting a Kv9.2 gene in a host cell. The nucleic acid construct comprises: a) a non-homologous replacement portion; b) a first homology region located upstream of the non-homologous replacement portion, the first homology region having a nucleotide sequence with substantial identity to a first Kv9.2 gene sequence; and c) a second homology region located downstream of the non-homologous replacement portion, the second homology region having a nucleotide sequence with substantial identity to a second Kv9.2 gene sequence, the second Kv9.2 gene sequence having a location downstream of the first Kv9.2 gene sequence in a naturally occurring endogenous Kv9.2 gene. Additionally, the first and second homology regions are of sufficient length for homologous recombination between the nucleic acid construct and an endogenous Kv9.2 gene in a host cell when the nucleic acid molecule is introduced into the host cell. In a preferred embodiment, the non-homologous replacement portion comprises an expression reporter, preferably including lacZ and a positive selection expression cassette, preferably including a neomycin phosphotransferase gene operatively linked to a regulatory element(s).

Preferably, the first and second Kv9.2 gene sequences are derived from SEQ ID No. 1, SEQ ID No.2 or SEQ ID NO: 4, or a homologue, variant or derivative thereof.

Another aspect pertains to recombinant vectors into which the nucleic acid construct described above has been incorporated. Yet another aspect pertains to host cells into which the nucleic acid construct has been introduced to thereby allow homologous recombination between the nucleic acid construct and an endogenous Kv9.2 gene of the host cell, resulting in functional disruption of the endogenous Kv9.2 gene. The host cell can be a mammalian cell that normally expresses Kv9.2 from the liver, brain, spleen or heart, or a pluripotent cell, such as a mouse embryonic stem cell. Further development of an embryonic stem cell into which the nucleic acid construct has been introduced and homologously recombined with the endogenous Kv9.2 gene produces a transgenic nonhuman animal having cells that are descendant from the embryonic stem cell and thus carry the Kv9.2 gene disruption in their genome. Animals that carry the Kv9.2 gene disruption in their germline can then be selected and bred to produce animals having the Kv9.2 gene disruption in all somatic and germ cells. Such mice can then be bred to homozygosity for the Kv9.2 gene disruption.

Antibodies

The term “antibody” as used here, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. The antibodies and fragments thereof may be humanised antibodies, for example as described in EP-A-239400. Furthermore, antibodies with fully human variable regions (or their fragments), for example, as described in U.S. Pat. Nos. 5,545,807 and 6,075,181 may also be used. Neutralizing antibodies, i.e., those which inhibit biological activity of the substance amino acid sequences, are especially preferred for diagnostics and therapeutics.

Antibodies may be produced by standard techniques, such as by immunisation or by using a phage display library.

A polypeptide or peptide may be used to develop an antibody by known techniques. Such an antibody may be capable of binding specifically to the Kv9.2 protein or homologue, fragment, etc.

If polyclonal antibodies are desired, a selected mammal (e.g., mouse, rabbit, goat, horse, etc.) may be immunised with an immunogenic composition comprising a Kv9.2 polypeptide or peptide. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance amino acid sequence is administered to immunologically compromised individuals for the purpose of stimulating systemic defence.

Serum from the immunised animal is collected and treated according to known procedures. If serum containing polyclonal antibodies to an epitope obtainable from a polypeptide contains antibodies to other antigens, the polyclonal antibodies can be purified by immunoaffinity chromatography. Techniques for producing and processing polyclonal antisera are known in the art. In order that such antibodies may be made we also provide Kv9.2 amino acid sequences or fragments thereof haptenised to another amino acid sequence for use as immunogens in animals or humans.

Monoclonal antibodies directed against epitopes obtainable from a Kv9.2 polypeptide can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the trioma technique, the human B-cell hybridoma technique (Kosbor et al. (1983) Immunol Today 4:72, Cote et al. (1983) Proc Natl Acad Sci 80:2026-2030) and the EBV-hybridoma technique (Cole et al., Monoclonal Antibodies and Cancer Therapy, pp. 77-96, Alan R. Liss, Inc., 1985).

In addition, techniques developed for the production of “chimeric antibodies”, the splicing of mouse antibody genes to human antibody genes to obtain a molecule with appropriate antigen specificity and biological activity can be used (Morrison et al. (1984) Proc Natl Acad Sci 81:6851-6855; Neuberger et al. (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from a Kv9.2 polypeptide or peptide are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al. (1989, Proc Natl Acad Sci 86: 3833-3837), and Winter G and Milstein C (1991; Nature 349:293-299).

Antibody fragments which contain specific binding sites for the polypeptide or peptide may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256: 1275-128 1).

Techniques for the production of single chain antibodies (U.S. Pat. No. 4,946,778) can also be adapted to produce single chain antibodies to Kv9.2 polypeptides. Also, transgenic mice, or other organisms including other mammals, may be used to express humanized antibodies.

The above-described antibodies may be employed to isolate or to identify clones expressing the polypeptide or to purify the polypeptides by affinity chromatography.

Antibodies against Kv9.2 subunit polypeptides may also be employed to modulate blood glucose for the treatment of diseases including, Type I and Type II diabetes, hyperinsulinaemia, hyperinsulinism, insulin resistance, complications of diabetes including diabetes associated vascular disease, diabetes associated renal disease and diabetes associated neuropathy, and the treatment of hypoglycaemia, hyperlipoidemia (be they HDL, LDL or VLDL), and dyslipoidemia, whether primary in origin or secondary to diabetes, hyper/hypothyroidism, acromegaly, liver failure, renal failure, pancreatic tumours, pancreatitis and alcohol induced hypoglycaemia.

Diagnostic Assays

We further describe the use of Kv9.2 subunit polynucleotides and polypeptides (as well as homologues, variants and derivatives thereof) for use in diagnosis as diagnostic reagents or in genetic analysis. Nucleic acids complementary to or capable of hybridising to Kv9.2 subunit nucleic acids (including homologues, variants and derivatives), as well as antibodies against Kv9.2 polypeptides are also useful in such assays.

Detection of a mutated form of the Kv9.2 subunit gene associated with a dysfunction will provide a diagnostic tool that can add to or define a diagnosis of a disease or susceptibility to a disease which results from under-expression, over-expression or altered expression of Kv9.2 subunit. Individuals carrying mutations in the Kv9.2 subunit gene (including control sequences) may be detected at the DNA level by a variety of techniques.

For example, DNA may be isolated from a patient and the DNA polymorphism pattern of Kv9.2 determined. The identified pattern is compared to controls of patients known to be suffering from a disease associated with over-, under- or abnormal expression of Kv9.2. Patients expressing a genetic polymorphism pattern associated with associated disease may then be identified. Genetic analysis of the Kv9.2 subunit gene may be conducted by any technique known in the art. For example, individuals may be screened by determining DNA sequence of a Kv9.2 allele, by RFLP or SNP analysis, etc. Patients may be identified as having a genetic predisposition for a disease associated with the over-, under-, or abnormal expression of Kv9.2 by detecting the presence of a DNA polymorphism in the gene sequence for Kv9.2 or any sequence controlling its expression.

Patients so identified can then be treated to prevent the occurrence of Kv9.2 associated disease, or more aggressively in the early stages of Kv9.2 associated disease to prevent the further occurrence or development of the disease. Kv9.2 associated diseases include Type I and Type II diabetes, hyperinsulinaemia, hyperinsulinism, insulin resistance, complications of diabetes including diabetes associated vascular disease, diabetes associated renal disease and diabetes associated neuropathy, and the treatment of hypoglycaemia, hyperlipoidemia (be they HDL, LDL or VLDL), and dyslipoidemia, whether primary in origins or secondary to diabetes, hyper/hypothyroidism, acromegaly, liver failure, renal failure, pancreatic tumours, pancreatitis and alcohol induced hypoglycaemia. Also for treatment of neurotic disorders including anxiety, anxiety disorders, anxiety-related behaviour and generalized anxiety disorder, panic disorder, agoraphobia, social phobia, obsessive-compulsive disorder, posttraumatic stress disorder, acute stress disorder, and those panic disorders list in DSM-IV, and depression.

We further disclose a kit for the identification of a patient's genetic polymorphism pattern associated with Kv9.2 associated disease. The kit includes DNA sample collecting means and means for determining a genetic polymorphism pattern, which is then compared to control samples to determine a patient's susceptibility to Kv9.2 associated disease. Kits for diagnosis of a Kv9.2 associated disease comprising Kv9.2 polypeptide and/or an antibody against such a polypeptide (or fragment of it) are also provided.

Nucleic acids for diagnosis may be obtained from a subject's cells, such as from blood, urine, saliva, tissue biopsy or autopsy material. In a preferred embodiment, the DNA is obtained from blood cells obtained from a finger prick of the patient with the blood collected on absorbent paper. In a further preferred embodiment, the blood will be collected on an AmpliCard™ (University of Sheffield, Department of Medicine and Pharmacology, Royal Hallamshire Hospital, Sheffield, England S10 2JF).

The DNA may be used directly for detection or may be amplified enzymatically by using PCR or other amplification techniques prior to analysis. Oligonucleotide DNA primers that target the specific polymorphic DNA region within the genes of interest may be prepared so that in the PCR reaction amplification of the target sequences is achieved. RNA or cDNA may also be used as templates in similar fashion. The amplified DNA sequences from the template DNA may then be analyzed using restriction enzymes to determine the genetic polymorphisms present in the amplified sequences and thereby provide a genetic polymorphism profile of the patient. Restriction fragments lengths may be identified by gel analysis. Alternatively, or in conjunction, techniques such as SNP (single nucleotide polymorphisms) analysis may be employed.

Deletions and insertions can be detected by a change in size of the amplified product in comparison to the normal genotype. Point mutations can be identified by hybridizing amplified DNA to labeled Kv9.2 subunit nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al, Science (1985)230: 1242. Sequence changes at specific locations may also be revealed by nuclease protection assays, such as RNAse and S1 protection or the chemical cleavage method. See Cotton et al., Proc Natl Acad Sci USA (1985) 85: 4397-4401. In another embodiment, an array of oligonucleotides probes comprising the Kv9.2 subunit nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al., Science, Vol 274, pp 610-613 (1996)).

Single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control nucleic acids may be denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

The diagnostic assays offer a process for diagnosing or determining a susceptibility to diseases such as anxiety or diabetes by detection of mutation in the Kv9.2 subunit gene by the methods described.

The presence of Kv9.2 subunit polypeptides and nucleic acids may be detected in a sample. Thus, infections and diseases as listed above can be diagnosed by methods comprising determining from a sample derived from a subject an abnormally decreased or increased level of the Kv9.2 subunit polypeptide or Kv9.2 subunit mRNA. The sample may comprise a cell or tissue sample from an organism suffering or suspected to be suffering from a disease associated with increased, reduced or otherwise abnormal Kv9.2 subunit expression, including spatial or temporal changes in level or pattern of expression. The level or pattern of expression of Kv9.2 in an organism suffering from or suspected to be suffering from such a disease may be usefully compared with the level or pattern of expression in a normal organism as a means of diagnosis of disease.

In general therefore, we disclose a method of detecting the presence of a nucleic acid comprising a Kv9.2 subunit nucleic acid in a sample, by contacting the sample with at least one nucleic acid probe which is specific for said nucleic acid and monitoring said sample for the presence of the nucleic acid. For example, the nucleic acid probe may specifically bind to the Kv9.2 subunit nucleic acid, or a portion of it, and binding between the two detected; the presence of the complex itself may also be detected. Furthermore, we disclose a method of detecting the presence of a Kv9.2 subunit polypeptide by contacting a cell sample with an antibody capable of binding the polypeptide and monitoring said sample for the presence of the polypeptide. This may conveniently be achieved by monitoring the presence of a complex formed between the antibody and the polypeptide, or monitoring the binding between the polypeptide and the antibody. Methods of detecting binding between two entities are known in the art, and include FRET (fluorescence resonance energy transfer), surface plasmon resonance, etc.

Decreased or increased expression can be measured at the RNA level using any of the methods well known in the art for the quantitation of polynucleotides, such as, for example, PCR, RT-PCR, RNAse protection, Northern blotting and other hybridization methods. Assay techniques that can be used to determine levels of a protein, such as a Kv9.2 subunit, in a sample derived from a host are well-known to those of skill in the art. Such assay methods include radioimmunoassays, competitive-binding assays, Western Blot analysis and ELISA assays.

This disclosure also relates to a diagnostic kit for a disease or susceptibility to a disease (including an infection), for example, anxiety or diabetes. The diagnostic kit comprises a Kv9.2 subunit polynucleotide or a fragment thereof; a complementary nucleotide sequence; a Kv9.2 subunit polypeptide or a fragment thereof, or an antibody to a Kv9.2 subunit polypeptide.

Chromosome Assays

The Kv9.2 nucleotide sequences are also valuable for chromosome identification. The sequence is specifically targeted to and can hybridize with a particular location on an individual human chromosome. As described above, human Kv9.2 subunit is found to map to Homo sapiens chromosome 8q22.

The mapping of relevant sequences to chromosomes is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

Prophylactic and Therapeutic Methods

We provide methods of treating an abnormal conditions related to both an excess of and insufficient amounts of Kv9.2 subunit activity.

If the activity of Kv9.2 subunit is in excess, several approaches are available. One approach comprises administering to a subject an inhibitor compound (antagonist) as hereinabove described along with a pharmaceutically acceptable carrier in an amount effective to inhibit activation by blocking binding of ligands to the Kv9.2 subunit, or by inhibiting a second signal, and thereby alleviating the abnormal condition.

In another approach, soluble forms of Kv9.2 subunit polypeptides still capable of binding the ligand in competition with endogenous Kv9.2 subunit may be administered. Typical embodiments of such competitors comprise fragments of the Kv9.2 subunit polypeptide.

In still another approach, expression of the gene encoding endogenous Kv9.2 subunit can be inhibited using expression blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of Kv9.2 subunit and its activity, several approaches are also available. One approach comprises administering to a subject a therapeutically effective amount of a compound which activates Kv9.2 containing ion channel, i.e., an agonist or opener as described above, in combination with a pharmaceutically acceptable carrier, to thereby alleviate the abnormal condition. Alternatively, gene therapy may be employed to effect the endogenous production of Kv9.2 subunit by the relevant cells in the subject. For example, a Kv9.2 polynucleotide may be engineered for expression in a replication defective retroviral vector, as discussed above. The retroviral expression construct may then be isolated and introduced into a packaging cell transduced with a retroviral plasmid vector containing RNA encoding a Kv9.2 polypeptide such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

Formulation and Administration

Peptides, such as the soluble form of Kv9.2 subunit polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. We further disclose to pharmaceutical packs and kits comprising one or more containers filled with one or more of the ingredients of the aforementioned compositions.

Kv9.2 polypeptides and other compounds may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localize, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Pharmaceutical Compositions

We also provide a pharmaceutical composition comprising administering a therapeutically effective amount of the Kv9.2 polypeptide, polynucleotide, peptide, vector or antibody and optionally a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition described here may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Vaccines

Another embodiment relates to a method for inducing an immunological response in a mammal which comprises inoculating the mammal with the Kv9.2 subunit polypeptide, or a fragment thereof, adequate to produce antibody and/or T cell immune response to protect said animal from abnormal blood glucose levels as a result of, but not limited to, Type I and Type II diabetes, hyperinsulinaemia, hyperinsulinism, insulin resistance, complications of diabetes including diabetes associated vascular disease, diabetes associated renal disease and diabetes associated neuropathy, and the treatment of hypoglycaemia, hyperlipoidemia (be they HDL, LDL or VLDL), and dyslipoidemia, whether primary in origins or secondary to diabetes, hyper/hypothyroidism, acromegaly, liver failure, renal failure, pancreatic tumours, pancreatitis and alcohol induced hypoglycaemia, among others.

Yet another embodiment relates to a method of inducing immunological response in a mammal which comprises delivering a Kv9.2 polypeptide via a vector directing expression of a Kv9.2 subunit polynucleotide in vivo in order to induce such an immunological response to produce antibody to protect said animal from diseases.

A further embodiment relates to an immunological/vaccine formulation (composition) which, when introduced into a mammalian host, induces an immunological response in that mammal to a Kv9.2 polypeptide wherein the composition comprises a Kv9.2 polypeptide or Kv9.2 gene. The vaccine formulation may further comprise a suitable carrier.

Since the Kv9.2 polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Vaccines may be prepared from one or more Kv9.2 polypeptides or peptides.

The preparation of vaccines which contain an immunogenic polypeptide(s) or peptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydrooxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The Kv9.2 polypeptides may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The pharmaceutical and vaccine compositions may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution, a parenteral route where delivery is by an injectable form, such as, for example, an intravenous, intramuscular or subcutaneous route.

The term “co-administered” means that the site and time of administration of each of for example, the Kv9.2 polypeptide and an additional entity such as adjuvant are such that the necessary modulation of the immune system is achieved. Thus, whilst the polypeptide and the adjuvant may be administered at the same moment in time and at the same site, there may be advantages in administering the polypeptide at a different time and to a different site from the adjuvant. The polypeptide and adjuvant may even be delivered in the same delivery vehicle—and the polypeptide and the antigen may be coupled and/or uncoupled and/or genetically coupled and/or uncoupled.

The Kv9.2 polypeptide, polynucleotide, peptide, nucleotide, antibody and optionally an adjuvant may be administered separately or co-administered to the host subject as a single dose or in multiple doses.

The vaccine composition and pharmaceutical compositions may be administered by a number of different routes such as injection (which includes parenteral, subcutaneous and intramuscular injection) intranasal, mucosal, oral, intra-vaginal, urethral or ocular administration.

The vaccines and pharmaceutical compositions may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides, such suppositories may be formed from mixtures containing the active ingredient in the range of 0.5% to 10%, may be 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Further Aspects

Further aspects and embodiments of the invention are now set out in the following numbered Paragraphs, it is to be understood that the invention encompasses these aspects:

Paragraph 1. A Kv9.2 polypeptide comprising the amino acid sequence shown in SEQ ID NO. 3 or SEQ ID NO: 5, or a homologue, variant or derivative thereof.

Paragraph 2. A nucleic acid encoding a polypeptide according to Paragraph 1.

Paragraph 3. A nucleic acid according to Paragraph 2, comprising the nucleic acid sequence shown in SEQ ID No. 1, SEQ ID No.2 or SEQ ID NO: 4, or a homologue, variant or derivative thereof.

Paragraph 4. A polypeptide comprising a fragment of a polypeptide according to Paragraph 1.

Paragraph 5. A polypeptide according to Paragraph 3 which comprises one or more regions which are homologous between SEQ ID No. 3 and SEQ ID No. 5, or which comprises one or more regions which are heterologous between SEQ ID No. 3 and SEQ ID No. 5.

Paragraph 6. A nucleic acid encoding a polypeptide according to Paragraph 4 or 5.

Paragraph 7. A vector comprising a nucleic acid according to Paragraph 2, 3, or 6.

Paragraph 8. A host cell comprising a nucleic acid according to Paragraph 2, 3, or 6, or vector according to Paragraph 7.

Paragraph 9. A transgenic non-human animal comprising a nucleic acid according to Paragraph 2, 3 or 6, or a vector according to Paragraph 7.

Paragraph 10. A transgenic non-human animal according to Paragraph 9 which is a mouse.

Paragraph 11. Use of a polypeptide according to Paragraph 1, 4 or 5 in a method of identifying a compound which is capable of interacting specifically with a Kv9.2 subunit.

Paragraph 12. Use of a transgenic non-human animal according to Paragraph 9 or 10 in a method of identifying a compound which is capable of interacting specifically with a Kv9.2 subunit.

Paragraph 13. A method for identifying an antagonist of a Kv9.2 containing ion channel, the method comprising contacting a cell which expresses Kv9.2 with a candidate compound and determining whether the kinetics and conductance of the channel is altered.

Paragraph 14. A method for identifying a compound capable of increasing the conductance level of the channel or modulating the current kinetics of the channel which method comprises contacting a cell which expresses a Kv9.2 containing ion channel with a candidate compound.

Paragraph 15. A method of identifying a compound capable of binding to a Kv9.2 polypeptide, the method comprising contacting a Kv9.2 polypeptide with a candidate compound and determining whether the candidate compound binds to the Kv9.2 polypeptide.

Paragraph 16. A compound identified by a method according to any of Paragraphs 11 to 15.

Paragraph 17. A compound capable of binding specifically to a polypeptide according to Paragraph 1, 4 or 5.

Paragraph 18. Use of a polypeptide according to Paragraph 1, 4 or 5, or part thereof or a nucleic acid according to Paragraph 2, 3 or 6, in a method for producing antibodies.

Paragraph 19. An antibody capable of binding specifically to a polypeptide according to Paragraph 1, 4 or 5, or part thereof or a polypeptide encoded by a nucleotide according to Paragraph 2, 3 or 6, or part thereof.

Paragraph 20. A pharmaceutical composition comprising any one or more of the following: a polypeptide according to Paragraph 1, 4 or 5, or part thereof; a nucleic acid according to Paragraph 2, 3 or 6, or part thereof, a vector according to Paragraph 7; a cell according to Paragraph 8; a compound according to Paragraph 16 or 17; and an antibody according to Paragraph 19, together with a pharmaceutically acceptable carrier or diluent.

Paragraph 21. A vaccine composition comprising any one or more of the following: a polypeptide according to Paragraph 1, 4 or 5, or part thereof, a nucleic acid according to Paragraph 2, 3 or 6, or part thereof; a vector according to Paragraph 7; a cell according to Paragraph 8; a compound according to Paragraph 16 or 17; and an antibody according to Paragraph 19.

Paragraph 22. A diagnostic kit for a disease or susceptibility to a disease comprising any one or more of the following: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19.

Paragraph 23. A method of treating a patient suffering from a disease associated with enhanced activity of a Kv9.2 containing ion channel, which method comprises administering to the patient an antagonist of Kv9.2 containing ion channel.

Paragraph 24. A method of treating a patient suffering from a disease associated with reduced activity of a Kv9.2 containing ion channel, which method comprises administering to the patient an agonist of Kv9.2 containing ion channel.

Paragraph 25. A method according to Paragraph 23 or 24, in which the Kv9.2 subunit comprises a polypeptide having the sequence shown in SEQ ID NO: 3 or SEQ ID NO: 5.

Paragraph 26. A method for treating and/or preventing a disease in a patient, which comprises the step of administering any one or more of the following to the patient: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; an antibody according to Paragraph 19; a pharmaceutical composition according to Paragraph 20; and a vaccine according to Paragraph 20.

Paragraph 27. An agent comprising a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and/or an antibody according to Paragraph 19, said agent for use in a method of treatment or prophylaxis of disease.

Paragraph 28. Use of a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19, for the preparation of a pharmaceutical composition for the treatment or prophylaxis of a disease.

Paragraph 29. A non-human transgenic animal, characterised in that the transgenic animal comprises an altered Kv9.2 gene.

Paragraph 30. A non-human transgenic animal according to Paragraph 29, in which the alteration is selected from the group consisting of: a deletion of Kv9.2, a mutation in Kv9.2 resulting in loss of function, introduction of an exogenous gene having a nucleotide sequence with targeted or random mutations into Kv9.2, introduction of an exogenous gene from another species into Kv9.2, and a combination of any of these.

Paragraph 31. A non-human transgenic animal having a functionally disrupted endogenous Kv9.2 gene, in which the transgenic animal comprises in its genome and expresses a transgene encoding a heterologous Kv9.2 protein.

Paragraph 32. A nucleic acid construct for functionally disrupting a Kv9.2 gene in a host cell, the nucleic acid construct comprising: (a) a non-homologous replacement portion; (b) a first homology region located upstream of the non-homologous replacement portion, the first homology region having a nucleotide sequence with substantial identity to a first Kv9.2 gene sequence; and (c) a second homology region located downstream of the non-homologous replacement portion, the second homology region having a nucleotide sequence with substantial identity to a second Kv9.2 gene sequence, the second Kv9.2 gene sequence having a location downstream of the first Kv9.2 gene sequence in a naturally occurring endogenous Kv9.2 gene.

Paragraph 33. A process for producing a Kv9.2 polypeptide, the method comprising culturing a host cell according to Paragraph 8 under conditions in which a nucleic acid encoding a Kv9.2 polypeptide is expressed.

Paragraph 34. A method of detecting the presence of a nucleic acid according to Paragraph 2, 3 or 6 in a sample, the method comprising contacting the sample with at least one nucleic acid probe which is specific for said nucleic acid and monitoring said sample for the presence of the nucleic acid.

Paragraph 35. A method of detecting the presence of a polypeptide according to Paragraph 1, 4 or 5 in a sample, the method comprising contacting the sample with an antibody according to Paragraph 19 and monitoring said sample for the presence of the polypeptide.

Paragraph 36. A method of diagnosis of a disease or syndrome caused by or associated with increased, decreased or otherwise abnormal expression of Kv9.2 subunit, the method comprising the steps of: (a) detecting the level or pattern of expression of Kv9.2 subunit in an animal suffering or suspected to be suffering from such a disease, and (b) comparing the level or pattern of expression with that of a normal animal.

### EXAMPLES

### Example 1

### Transgenic Kv9.2 Knock-Out Mouse: Construction of Kv9.2 Gene Targeting Vector

The Kv9.2 gene was identified bio-informatically using homology searches of genome databases. A 226 kb genomic contig was assembled from various databases. This contig provided sufficient flanking sequence information to enable the design of homologous arms to clone into the targeting vector.

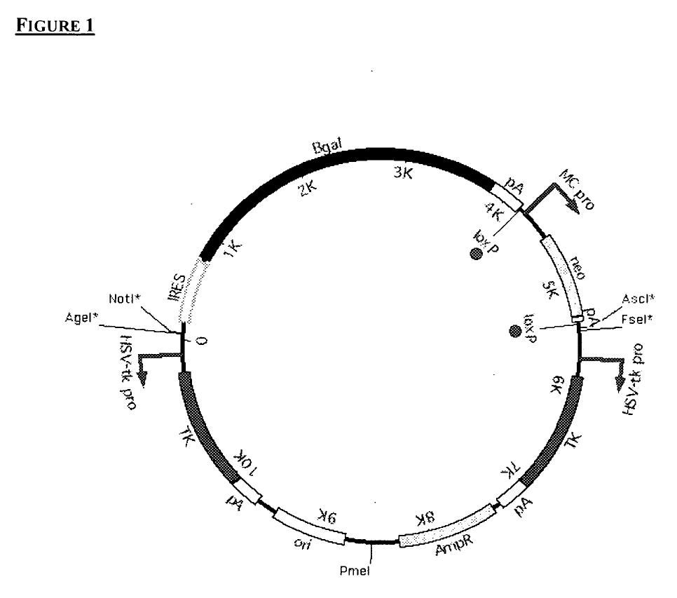

The murine Kv9.2 gene has 1 coding exon. The targeting strategy is designed to remove the majority of the coding sequence. A 1.7 kb 5′ homologous arm and a 4.0 kb 3′ homologous arm flanking the region to be deleted are amplified by PCR and the fragments are cloned into the targeting vector. The 5′ end of each oligonucleotide primer used to amplify the arms is synthesised to contain a different recognition site for a rare-cutting restriction enzyme, compatible with the cloning sites of the vector polylinkers and absent from the arms themselves. In the case of Kv9.2, the primers are designed as listed in the primer table below, with 5′ arm cloning sites of AgeI/NotI and 3′ arm cloning sites of AscI/FseI (the structure of the targeting vector used, including the relevant restriction sites, is shown in FIG. 1).

In addition to the arm primer pairs (5′armF/5′armR) and (3′armF/3′armR), further primers specific to the Kv9.2 locus are designed for the following purposes: 5′ and 3′ probe primer pairs (5′prF/5′prR and 3′prF/3′prR) to amplify two short 150-300 bp fragments of non-repetitive genomic DNA external to and extending beyond each arm, to allow Southern analysis of the targeted locus, in isolated putative targeted clones, a mouse genotyping primer pair (hetF and hetR) which allows differentiation between wild-type, heterozygote and homozygous mice, when used in a multiplex PCR with a vector specific primer, in this case, Asc403, and lastly, a target screening primer (5′scr) which anneals upstream of the end of the 5′ arm region, and which produces a target event specific 2.0 kb amplimer when paired with a primer specific to the 5′ end of the vector (TK5IBLMNL), in this case DR1. This amplimer can only be derived from template DNA from cells where the desired genomic alteration has occurred and allows the identification of correctly targeted cells from the background of clones containing randomly integrated copies of the vector. The location of these primers and the genomic structure of the regions of the Kv9.2 locus used in the targeting strategy is shown in SEQ ID NO: 19.

The position of the homology arms is chosen to functionally disrupt the Kv9.2 gene. A targeting vector is prepared where the Kv9.2 region to be deleted is replaced with non-homologous sequences composed of an endogenous gene expression reporter (a frame independent lacZ gene) upstream of a selection cassette composed of a promoted neomycin phosphotransferase (neo) gene arranged in the same orientation as the Kv9.2 gene.

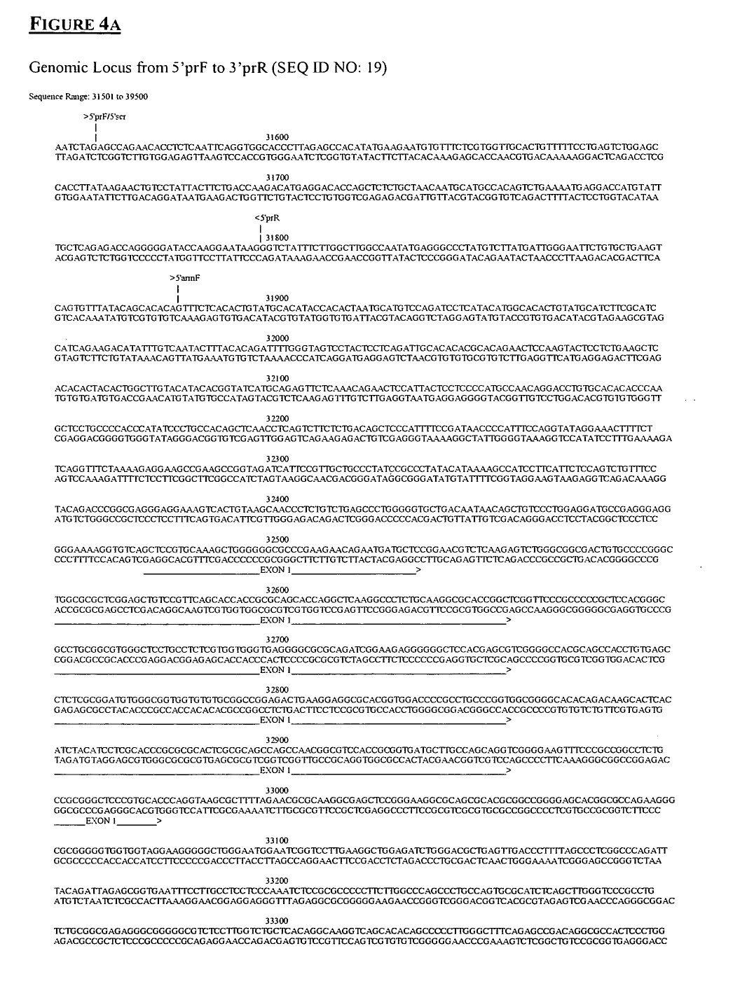

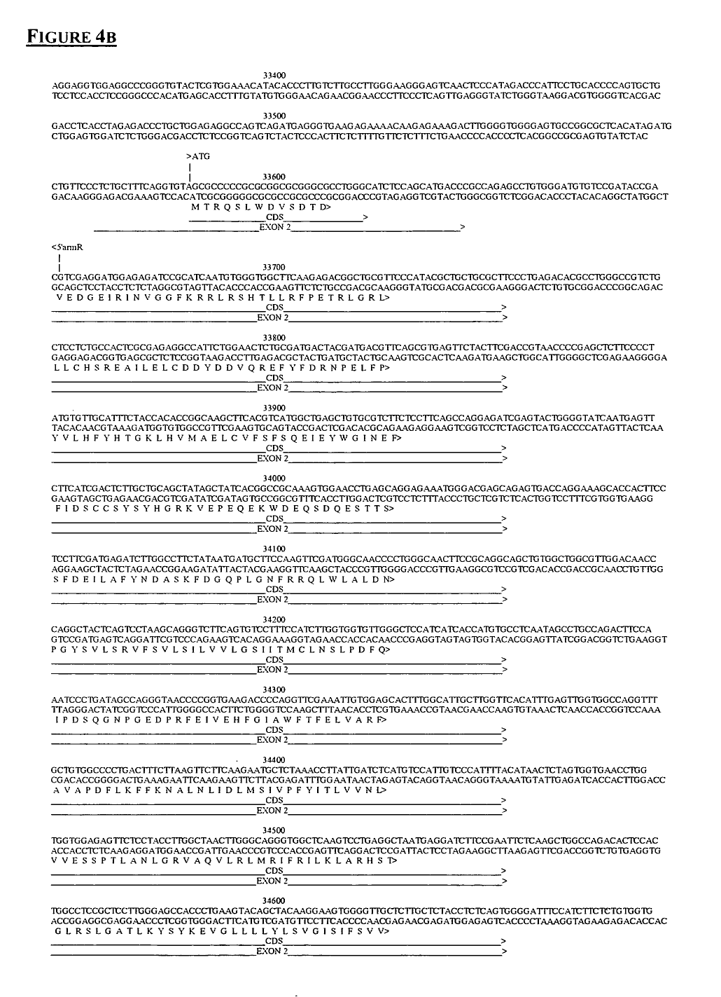

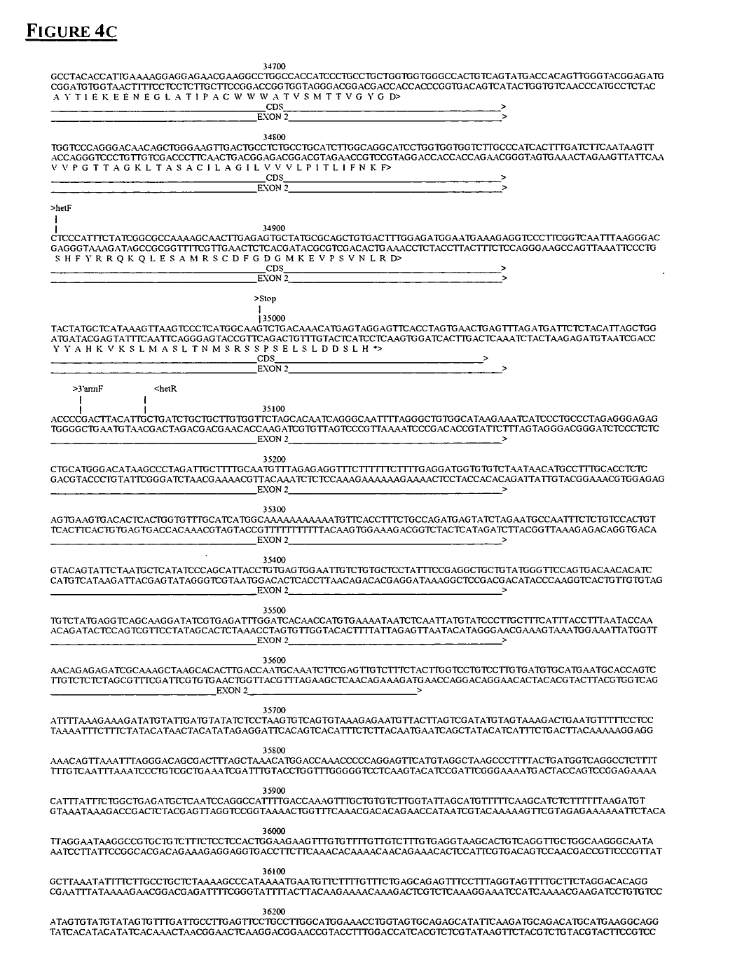

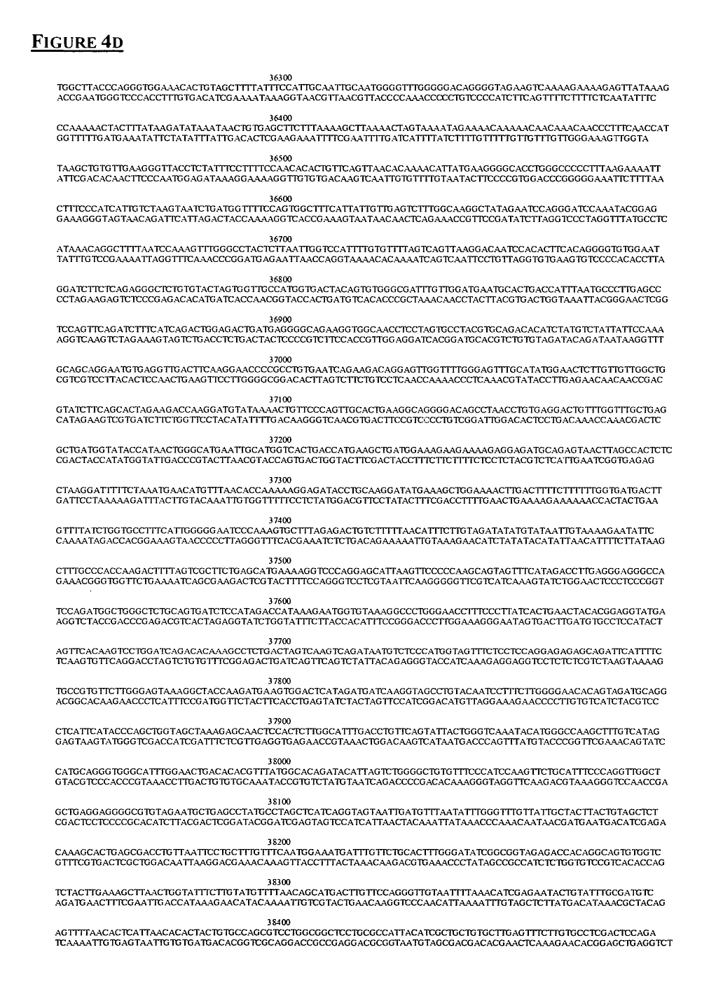

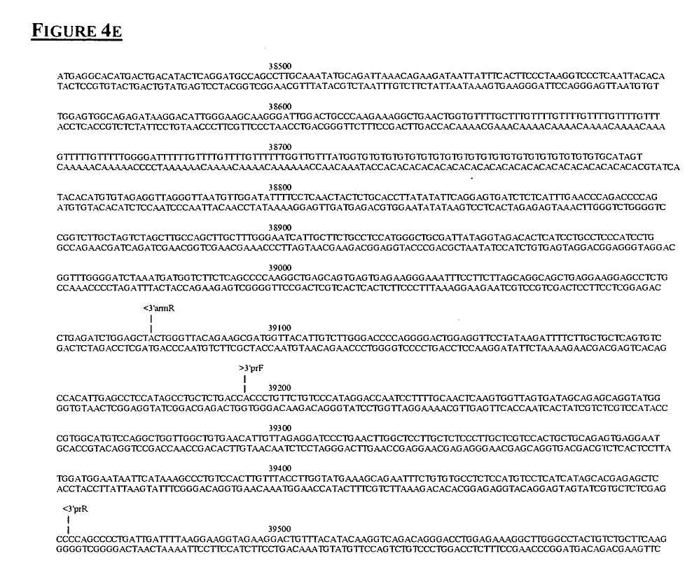

Once the 5′ and 3′ homology arms have been cloned into the targeting vector TK5IBLMNL (see FIG. 4), a large highly pure DNA preparation is made using standard molecular biology techniques. 20 μg of the freshly prepared endotoxin-free DNA is restricted with another rare-cutting restriction enzyme PmeI, present at a unique site in the vector backbone between the ampicillin resistance gene and the bacterial origin of replication. The linearized DNA is then precipitated and resuspended in 100 μl of Phosphate Buffered Saline, ready for electroporation.

24 hours following electroporation the transfected cells are cultured for 9 days in medium containing 200 μg/ml neomycin. Clones are picked into 96 well plates, replicated and expanded before being screened by PCR (using primers 5′prF and DR1, as described above) to identify clones in which homologous recombination has occurred between the endogenous Kv9.2 gene and the targeting construct. Positive clones can be identified at a rate of 1 to 5%. These clones are expanded to allow replicas to be frozen and sufficient high quality DNA to be prepared for Southern blot confirmation of the targeting event using the external 5′ and 3′ probes prepared as described above, all using standard procedures (Russ et al., Nature 2000 Mar. 2; 404(6773):95-99). When Southern blots of DNA digested with diagnostic restriction enzymes are hybridized with an external probe, homologously targeted ES cell clones are verified by the presence of a mutant band as well an unaltered wild-type band. For instance, wild-type genomic DNA digested with AflII will yield a band of 6.2 kb when hybridized with the 5′ external probe and 7.0 kb with the 3′ external probe, while similarly digested genomic DNA containing a targeted allele will yield a 17 kb knockout specific band in addition to the wild-type band.

### Example 2

### Transgenic Kv9.2 Knock-Out Mouse: Generation of Kv9.2 GPCR Deficient Mice

C57BL/6 female and male mice are mated and blastocysts are isolated at 3.5 days of gestation. 10-12 cells from a chosen clone are injected per blastocyst and 7-8 blastocysts are implanted in the uterus of a pseudopregnant F1 female. A litter of chimeric pups are born containing several high level (up to 100%) agouti males (the agouti coat colour indicates the contribution of cells descended from the targeted clone). These male chimeras are mated with female MF1 and 129 mice, and germline transmission is determined by the agouti coat colour and by PCR genotyping respectively.

PCR Genotyping is carried out on lysed tail clips, using the primers hetF and hetR with a third, vector specific primer (Asc403). This multiplex PCR allows amplification from the wild-type locus (if present) from primers hetF and hetR giving a 241 bp band. The site for hetF is deleted in the knockout mice, so this amplification will fail from a targeted allele. However, the Asc403 primer will amplify a 434 bp band from the targeted locus, in combination with the hetR primer which anneals to a region just inside the 3′ arm. Therefore, this multiplex PCR reveals the genotype of the litters as follows: wild-type samples exhibit a single 241 bp band; heterozygous DNA samples yield two bands at 241 bp and 434 bp; and the homozygous samples will show only the target specific 434 bp band.

TABLE 1
Kv9.2 Primer Sequences

| | | |
|---|---|---|
| musKv9.2 5′pr F | CTCTCAATTCAGGTGGCACCCTTAGAG - | Seq ID No. 6 |
| musKv9.2 5′pr R | CACAGAATTCCCAATCATAAGACATAG - | Seq ID No. 7 |
| musKv9.2 5′scr DR1 | CTCTCAATTCAGGTGGCACCCTTAGAG - | Seq ID No. 8 |
| musKv9.2 5′arm F Age | AaaaccggtATGTCCAGATCCTCATACATGGCACAC - | Seq ID No. 9 |
| musKv9.2 5′arm R Not | AaagcggccgcGACGTCGGTATCGGACACATCCCACAG - | Seq ID No. 10 |
| musKv9.2 3′arm F Asc | TttggcgcgccTTGCTGATCTGCTGCTTGTGGTTCTAG - | Seq ID No. 11 |
| musKv9.2 3′arm R Fse | AaaggccggccAATGTAACCATCGCTTCTGTAACCCAG - | Seq ID No. 12 |
| musKv9.2 3′pr F | AGCAGAGCAGGTATGGCGTGGCATGTC - | Seq ID No. 13 |
| musKv9.2 3′pr R | CTGGGGGAGCTCTCGTGCTATGATGAG - | Seq ID No. 14 |
| musKv9.2 hetF | CCCATTTCTATCGGCGCCAAAAGCAAC - | Seq ID No. 15 |
| musKv9.2 hetR a403 | GTGCTAGAACCACAAGCAGCAGATCAG - | Seq ID No. 16 |
| Asc403 | CAGCCGAACTGTTCGCCAGGCTCAAGG - | Seq ID No. 17 |
| DR1 | CATGCCGCCTGCGCCCTATTGATCATG - | Seq ID No. 18 |

### Example 3

### Biological Data: Gene Expression Patterns (Human RT-PCR)

Using RT-PCR on human tissues, expression of the gene was shown in the lung, brain, spleen and to a lesser extent, the prostate, liver, reproductive organs and muscle.

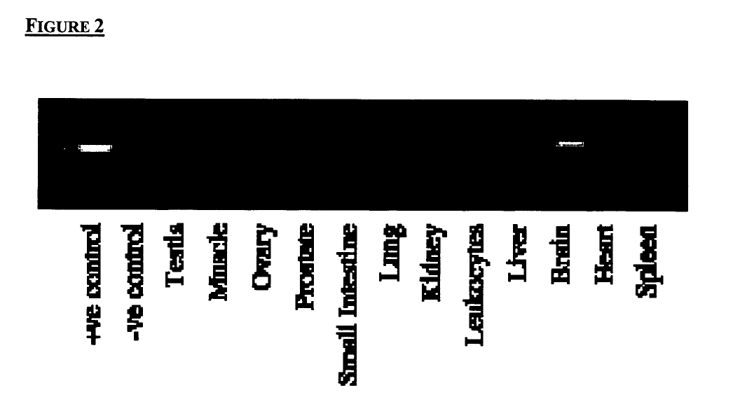

This is shown in FIG. 2.

### Example 4

### Biological Data: Gene Expression Pattern (Lac Z Stained Structures)

LacZ Staining

The X gal staining of dissected tissues is performed in the following manner.

Representative tissue slices are made of large organs. Whole small organs and tubes are sliced open, so fixative and stain will penetrate. Tissues are rinsed thoroughly in PBS (phosphate buffered saline) to remove blood or gut contents. Tissues are placed in fixative (PBS containing 2% formaldehyde, 0.2% glutaraldehyde, 0.02% NP40, 1 mM MgCl₂, Sodium deoxycholate 0.23 mM) for 30-45 minutes. Following three 5 minute washes in PBS, tissues are placed in Xgal staining solution (4 mMKFerrocyanide, 4 mMKFerrocyanide, 2 mM MgCl₂, 1 mg/mlX-gal in PBS) for 18 hours at 30 C. Tissues are PBS washed 3 times, postfixed for 24 hours in 4% formaldehyde, PBS washed again before storage in 70% ethanol.

To identify Xgal stained tissues, dehydrated tissues are wax embedded, and 7 um section sections cut, counterstained with 0.01% Safranin (9-10 min).

Using LacZ staining, Kv9.2 is found to be expressed in the brain and in particularly in the cortex, hippo campus, islands of calleja, ventate pallidum, central amydaloid nucleus (CeL), thalamic nuclei and cortex. In addition, evidence of staining is also seen in the heart, spleen, lung, and testis.

### Example 5

### Biological Data: Physiology/Biochemistry (Blood Glucose)

Blood glucose readings were taken from a blood sample taken from the tail vein of animals that had been fasted overnight (14-16 hours). Blood glucose was measured using a blood glucose monitor.

Animals with the Kv9.2 gene knocked out were examined and compared to litter mates without the knockout (wildtype). The knockout animals had a blood sugar level of 3.24±2.1 mmol/l compared to 4.69±0.38 mmol/l for wildtypes (P=0.006). Accordingly, Kv9.2 knockout animals display decreased blood glucose levels compared to wild type animals.

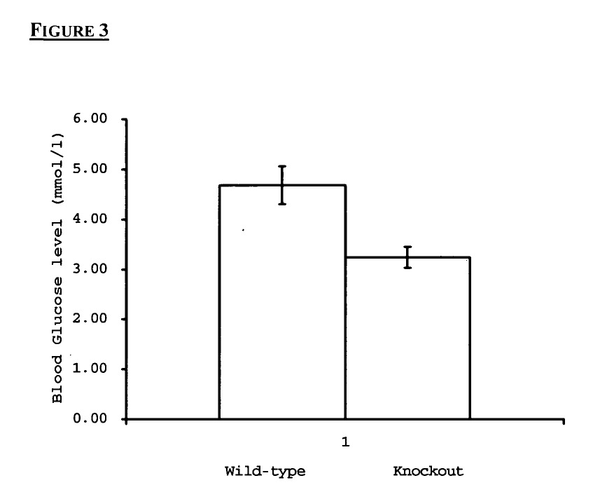

The results are shown in FIG. 3.

### Example 6

### Biological Data: Behavioural Analysis (Open Field Test)

Knockout and wild-type control mice were tested in an Open Field Test. See Carola, V., F. D'Olimpio, et al. (2002). “Evaluation of the elevated plus-maze and open-field tests for the assessment of anxiety-related behaviour in inbred mice.”.

Briefly, the mice are placed in the centre of a Perspex box with clear sides and the movement of the mice over a period of time is recorded on video. The mice are analysed for distance travelled, time spent moving and location of the mouse at any time. Control animals usually spend most of the time moving around the periphery of the arena with some crossing of the central zone. Variations from this normal pattern are recorded in particular the amount of time spent in the central areas of the arena, an increase of which can mean that the animal is less anxious.

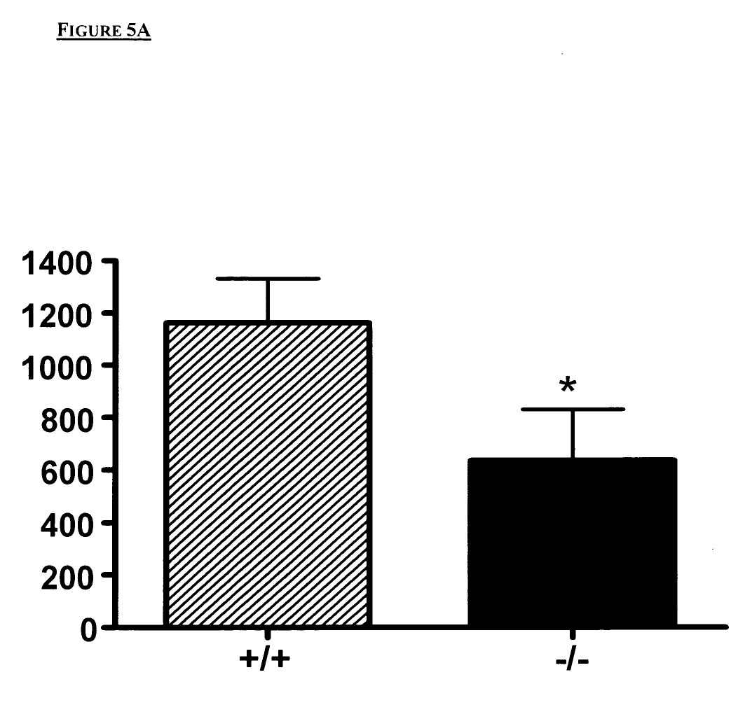

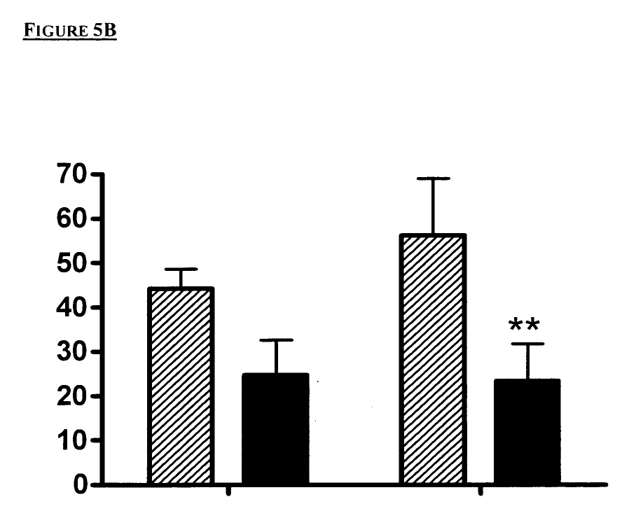

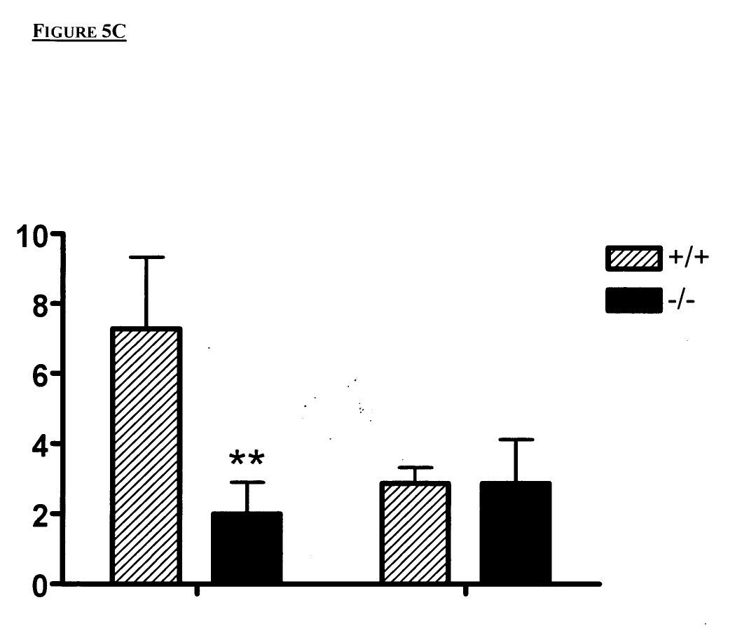

The movement of the mice over a period of time is recorded on video and analysed. The results show that knockout mice travelled less than the wild type control (WT 1161.39±170.8 cm; KO 636.84±193.62 p=0.05 ANOVA). (FIG. 5A). Breakdown of this data showed that the knockout mice had spent overall less time moving in both the peripheral areas and central area, i.e. they were immobile for longer (FIG. 5B), with a tendency to freeze compared with the movement of the wild type control mice. The Kv9.2 knockout mice therefore display increased immobility.

Time spent moving in central area WT 56.2±12.7 s; KO 23.3±8.5 s p=0.01 ANOVA, total time of test was 300 s, time spent moving in peripheral area WT 44.3±4.4; KO 24.7±7.9. The Kv9.2 knockout mice therefore furthermore display a decreased ambulation time, together with an increased peripheral permanence time.

The overall number of entries into the peripheral and centre zones i.e., the number of times the mice moved across the open field is also much reduced in the knockout mice (WT 7.3±2; KO 2±1 p<0.01 ANOVA) (FIG. 5C).

### Example 7

### Biological Data: Behavioural Analysis (Plus Maze)

Knockout and wild-type control mice were tested in a plus maze.

Briefly, anxiety in mice is measured using elevated plus maze and video tracking. This test exploits the conflict titrating the tendency of mice to explore a novel environment versus the aversive properties of a brightly lit open field, with added components of height and openness. Two alternating arms are closed with high dark walls and the two others are open. Mice prefer closed arms but will venture into the open ones. See Pellow et al. ‘Validation of open:closed arm entries in an elevated plus-maze as a measure of anxiety in the rat.’ (1985).

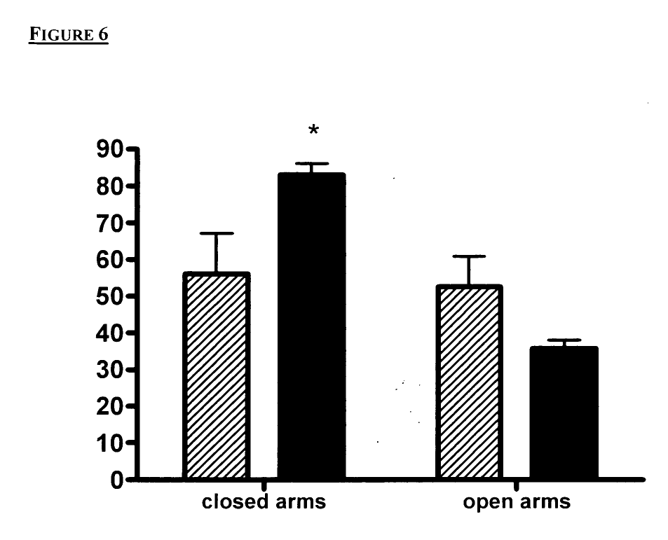

Although the knockout mice were recorded as moving a similar distance compared with the control wildtype mice, analysis of the data showed that they spent significantly longer in the closed arms (deemed to be safe) and made less entries into the open arms (which are deemed to be unsafe) compared with the control mice (FIG. 6).

This demonstrates that Kv9.2 knockout mice have an anxiety phenotype, and that accordingly Kv9.2 is involved in the disorder of anxiety.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments and that many modifications and additions thereto may be made within the scope of the invention. Indeed, various modifications of the described modes for carrying out the invention which are obvious to those skilled in molecular biology or related fields are intended to be within the scope of the claims. Furthermore, various combinations of the features of the following dependent claims can be made with the features of the independent claims without departing from the scope of the present invention.

Human Kv9.2 cDNA: SEQ ID No.1
GCCTCTCTGGGTGGGTGAGGGGCGCGCGGATCCGGAGAGGGGGCTCCGGG
AGCGGCGGGACCACGCAGCCACCTGTGAGCCTTCGGCAGCTGCGGGCGGC
GGCGGCGTACCCGGCCCGAGACGGGAGGAGACGCTCGGCGGCCCCCGCCC
GCCGGCCCGCCGGGCGCACACACTCGCACCCGCGCACGCACCGCCAGCAG
GCAGCGGCCACCGCCGCGATGCTCGCCCGCGGGTTGGGGAAGTTTCCCGC
CGGCCTCGGCCGCGGGCACCCGTGCTCCCAGGTGTAGCGCCCCCGCGCGG
CGCGGGCGGCCGGCGCCTCCAGCATGACCGGCCAGAGCCTGTGGGACGTG
TCGGAGGCTAACGTCGAGGACGGGGAGATCCGCATCAATGTGGGCGGCTT
CAAGAGGAGGCTGCGCTCGCACACGCTGCTGCGCTTCCCCGAGACGCGCC
TGGGCCCCTTGCTGCTCTGCCACTCGCGCGAGGCCATTCTGGAGCTCTGC
GATGACTACGACGACGTCCAGCGGGAGTTCTACTACGACCGCAACCCTGA
GCTCTTCCCCTACGTGCTGCATTTCTATCACACCGGCAAGCTTCACGTCA
TGGCTGAGCTATGTGTCTTCTCCTTCAGCCAGGAGATCGAGTACTGGGGC
ATCAACGAGTTCTTCATTGACTCCTGCTGCAGCTACAGCTACCATGGCCG
CAAAGTAGAGCCCGAGCAGGAGAAGTGGGACGAGCAGAGTGACCAGGAGA
GCACCACGTCTTCCTTCGATGAGATCCTTGCCTTCTACAACGACGCCTCC
AAGTTCGATGGGCAGCCCCTCGGCAACTTCCGCAGGCAGCTGTGGCTGGC
GCTGGACAACCCCGGCTACTCAGTGCTGAGCAGGGTCTTCAGCATCCTGT
CCATCCTGGTGGTGATGGGGTCCATCATCACCATGTGCCTCAATAGCCTG
CCCGATTTCCAAATCCCTGACAGCCAGGGCAACCCTGGCGAGGACCCTAG
GTTCGAAATCGTGGAGCACTTTGGCATTGCCTGGTTCACATTTGAGCTGG
TGGCCAGGTTTGCTGTGGCCCCTGACTTCCTCAAGTTCTTCAAGAATGCC
CTAAACCTTATTGACCTCATGTCCATCGTCCCCTTTTACATCACTCTGGT
GGTGAACCTGGTGGTGGAGAGCACACCTACTTTAGCCAACTTGGGCAGGG
TGGCCCAGGTCCTGAGGCTGATGCGGATCTTCCGCATCTTAAAGCTGGCC
AGGCACTCCACTGGCCTCCGCTCCCTGGGGGCCACTTTGAAATACAGCTA
CAAAGAAGTAGGGCTGCTCTTGCTCTACCTCTCCGTGGGGATTTCCATCT
TCTCCGTGGTGGCCTACACCATTGAAAAGGAGGAGAACGAGGGCCTGGCC
ACCATCCCTGCCTGCTGGTGGTGGGCTACCGTCAGTATGACCACAGTGGG
GTACGGGGATGTGGTCCCAGGGACCACGGCAGGAAAGCTGACTGCCTCTG
CCTGCATCTTGGCAGGCATCCTCGTGGTGGTCCTGCCCATCACCTTGATC
TTCAATAAGTTCTCCCACTTTTACCGGCGCCAAAAGCAACTTGAGAGTGC
CATGCGCAGCTGTGACTTTGGAGATGGAATGAAGGAGGTCCCTTCGGTCA
ATTTAAGGGACTATTATGCCCATAAAGTTAAATCCCTTATGGCAAGCCTG
ACGAACATGAGCAGGAGCTCACCAAGTGAACTCAGTTTAAATGATTCCCT
ACGTTAGCCGGGAGGACTTGTCACCCTCCACCCCACATTGCTGAGCTGCC
TCTTGTGCCTCTGGCACACCCCAGGCACCTTATGGTTATGGTGTAAGGAG
TATGCCCAGCCCCTGAGGGGAGAGATGCATGGGATATGCACCCAGGTTTC
TTTTACAGTTTTTAGAATCGTTTTTAGAGGGTGGTGTGTCTGACACCATG
CCTTTGCACCTTTCCATGAAATGACACTCACTGGTCTTTGCATCGTGGGC
ATAAAATGTTCACCTTTTTGCCAGATGAGTACACCCAGAATGCTAATTTT
TCTGTCCATCGTGTACGCTATTCTAGTGCTTGTGGCCCAGTACTGTCTAT
GAGTTGTCGTGCTCCTGTTTCTGAGGTTGTCGTGTGAGTTCTGTACAAAA
AGCCCCCACAAGTCGTCCAGTAGAAATGCATCTATGAGGTCAGCAAGGAT
ATGATGAGATTTTGCTCACAGTCATGTGAAAACAAAATCTCAGCTCTTTA
TCCATTGCTTTCACTTAGTTTTAGTACCAAAACAAAGAGAATGCAAAGTT
AAGCAGACTTGACCAATGCAAGTCTCTAAGTTGTTTTTATAAATGATCTG
TAGTTCCGTGGCTTGCATGGGTGCACCAATCATCTTTAGAACGATGTACA
CTGATGTTCATCTCATAAATGTCACTCTTTAGAGAATGTTACTTAGTTAA
ACATGCAGTGAAGATCGAATTTTTTTCCCAAGAACAGATGTGTTAGGGAG
AGGGGCTTCAGCTAAATAGTCCAAACCCTAGGGTGCTTAAAGCCAAGTTA
GTGCAGGCTGAGCCCCTTGGTTCACAGTCAAGCCTCCTTGTTTCCTAGGG
TGACTGTAGAGAAATGTATTTCCGGATGAGGTTTCTGATCTAGGCCATTT
GACCAAACTTTGCTGTGTCTAAGATATTAGCATGTTTTTGAAATATTTAT
TTTTTAAGATGTTTAGGAGTAAGGTCGTGTTGTCTTCCTCAACTAAAAAG
AAGTTTACTGTTGTATCGTCTCCCTGAGGTGAACGTTGTTGGGTTGCTAG
CAAGGGCAGTAGCTTAAATACTTTTGTTGCCTACTCTGAAAGCTCATCAA
ATGAGAGCCCTTTTATTTCCAAGCAGAATTTAGTCAGATAATTTTGCTTC
TAGGATATAGTATGTTGTATATGATGCTGTGATTGCCCTGGAGTTCCTGC
CATGACATGGAAACCTGGTGGTATGGAAGCATGTACTCAAAATATAGACG
TGCACGATGGTGGTGTGGCTTACCCAGGATGGAAACACTGCAGTTCTTAC
TTGCATTCCCACTGCCTTTCATGGGGGGTGACTGGGTAGAGGCCAGGAGA
AAGGAAAGAGTTGTAAAATAAAAAACTGCTAGTTCATAAAATGTCATAAA
AAATTGTAAACTTGAAAAGCTTAATGCTATTCAAAAGACCTTCAAGCTTC
CAAACTTGTATTGAAGGGAGACGACTGTTTCCTCCTCCAAAATGCTCCTG
CTCCTCTTGTTCGGTTAACCAGCACATAACATTGTGATGGGGAACCTGGG
TTCCTCTATAAGATAATTCTTCTCCATCATCTTTAAGGTAATCTGATGGT
TTTCCAGGTGGCTTTCATTATTGTTCCATCTTTGAAAAGGCAATAGAACC
CAGGGGTCTGAGCATGGAGCTATCCAGGGTTTTCATCCAAAGGTTGGGCC
TCTTCTTAAGAGGTCCTTTTGTGTTTCAGTTGATTGAAGATGATACTTAC
CTCATTGGAGGTGTGGCAAGGATCTTATCAGAAGGCTTTGTGTTCTTGTA
GTTGTCATGGCTACTACTGTGTGGGTGATTTATTGAATGAATTCACTAGC
CACTTGTGTCCTGGAGCCCCCAGTTCAAATCTTTCCATTGGACTGGAGGC
TTGTGGGAGGCTGGGAGGTGGCTGTCTCCTAGTGTCTACATCCGTGTCTC
TGAAGCATCAGGAAAAGTGAGATGACTTAGAGGCAACTGGGCACTGAATC
AGAGGAGCAGAGTTATTTTTCAGAATTTGCACATGGAACACTTAGATTTG
GCTGGTGCTTCCAGCCCTGGAAGGCATAACATTTACGGACTCATCCCCAG
CTGCACTGAAGGCAGGTGGTGGTACAGACTTATGAGGACGGATCAGTTTG
CCAAGGCTGATGGTATTGGGTCACTGAGCCTGGTATCCATGGCCGCTGAC
CAGGAAGCTTATGCAAAGTGGAAGCAAGGAACAAGGCAGAATAACTCAGT
CACTTTCATGAAGATTTTTCTAAACAAGAAGGCTTACCACCAAAAAAGAG
GTACCCTAGTGGTTACCCTTTGCAGATGTGAAAGCTGGAAAACTTGACTT
TTCTTTTTGGTAATGACTTGCATTTATCTGGTGCCTTTCGTTGGAGGAAT
CCCAACGTGCTTTAGAGACTATCTTTTTAACATCTCTTGTACATACATAT
ATACTTATATAAAATATTATCTTGCCCAACTGGACCTTTACTCACTTCTG
AGCATGAGAATGTCCCAATAGCATTGAGTTTTTCAAGTGGTGGTTTCAGA
TAAGTGGGAGAAAGAACAACCCGGCTGGCTTAAACCCTGGAGCTAATTCC
CACAAGGAATGTAGACTGAATGGTGACCCAGGGAGAAATAATCTTCCTCT
CCCCTAAAGTCTCACTAAGGTTTGAAGTTTACAGGTGCTCTCCACTGGGT
CTTTGATCGACCTTGCTAGATAACATCTAACTAAAAGCAGTTTCTTTTAG
TCCCTGAAGCTAACCAGGGAGAGTCAGGTTAATTTTCTGTAAAAATATGA
GGTGACATCTTTGGCAACCAGGCTGTCAGACTGACCTGTAAACCTCCTTT
AGGGGGACAGAGTAGAAACTGGAGATGACTTGTTTCCAGCTGTGAGCTTG
AGAGAAGTGTCACTCCCAGCATTTGAAGGTTATTGTTTTCAATGCCAGTG
GGCCAAATATATGGGCCAGGCTTTGATATCTGTGATGTGCATTTTGGAAG
TGCTGGGTTGGGAAGTGACACGTCTGTTGCACAAATGCATATTGGTTATA
GGTTTGTGTTTTCTGCCAAACCCCCACATTTCTCGGGTTTGTGAGTGAGG
AAGGGCATGTTGTAATGCCAAGCTGATTTGTAGCTCGTAAGGTAGTAATT
GGTATTTAACATTTGCATTTGTTATTTCTACTTATCTTAGCACTCAAATA
ATTGAACTACCTGCTAATTCTTGCCGCATTTCAAAGAAAATAAGTTGTTA
TGCACTTTGGGATAGTGGTGATCTGTACAGGCTGTGTGTTAGCTACTTGA
AGGCGTAACTGGTATTTCTTGTGTGTTTTAACAGCATGACTTCTTACAGA
GCTGTAATTTTTAAAATTGAGGATGCCATATTTGAGATGTCAGTTTTAAC
ACTCATTAACACACTACTGTGCAAGCATTGACACAGGCTGCACTG
Human Kv9.2 cDNA SEQ ID No.2
ATGACCGGCCAGAGCCTGTGGGACGTGTCGGAGGCTAACGTCGAGGACGG
GGAGATCCGCATCAATGTGGGCGGCTTCAAGAGGAGGCTGCGCTCGCACA
CGCTGCTGCGCTTCCCCGAGACGCGCCTGGGCCGCTTGCTGCTCTGCCAC
TCGCGCGAGGCCATTCTGGAGCTCTGCGATGACTACGACGACGTCCAGCG
GGAGTTCTACTTCGACCGCAACCCTGAGCTCTTCCCCTACGTGCTGCATT
TCTATCACACCGGCAAGCTTCACGTCATGGCTGAGCTATGTGTCTTCTCC
TTCAGCCAGGAGATCGAGTACTGGGGCATCAACGAGTTCTTCATTGACTC
CTGCTGCAGCTACAGCTACCATGGCCGCAAAGTAGAGCCCGAGCAGGAGA
AGTGGGACGAGCAGAGTGACCAGGAGAGCACCACGTCTTCCTTCGATGAG
ATCCTTGCCTTCTACAACGACGCCTCCAAGTTCGATGGGCAGCCCCTCGG
CAACTTCCGCAGGCAGCTGTGGCTGGCGCTGGACAACCCCGGCTACTCAG
TGCTGAGCAGGGTCTTCAGCATCCTGTCCATCCTGGTGGTGATGGGGTCC
ATCATCACCATGTGCCTCAATAGCCTGCCCGATTTCCAAATCCCTGACAG
CCAGGGCAACCCTGGCGAGGACCCTAGGTTCGAAATCGTGGAGCACTTTG
GCATTGCCTGGTTCACATTTGAGCTGGTGGCCAGGTTTGCTGTGGCCCCT
GACTTCCTCAAGTTCTTCAAGAATGCCCTAAACCTTATTGACCTCATGTC
CATCGTCCCCTTTTACATCACTCTGGTGGTGAACCTGGTGGTGGAGAGCA
CACCTACTTTAGCCAACTTGGGCAGGGTGGCCCAGGTCCTGAGGCTGATG
CGGATCTTCCGCATCTTAAAGCTGGCCAGGCACTCCACTGGCCTCCGCTC
CCTGGGGGCCACTTTGAAATACAGCTACAAAGAAGTAGGGCTGCTCTTGC
TCTACCTCTCCGTGGGGATTTCCATCTTCTCCGTGGTGGCCTACACCATT
GAAAAGGAGGAGAACGAGGGCCTGGCCACCATCCCTGCCTGCTGGTGGTG
GGCTACCGTCAGTATGACCACAGTGGGGTACGGGGATGTGGTCCCAGGGA
CCACGGCAGGAAAGCTGACTGCCTCTGCCTGCATCTTGGCAGGCATCCTC
GTGGTGGTCCTGCCCATCACCTTGATCTTCAATAAGTTCTCCCACTTTTA
CCGGCGCCAAAAGCAACTTGAGAGTGCCATGCGCAGCTGTGACTTTGGAG
ATGGAATGAAGGAGGTCCCTTCGGTCAATTTAAGGGACTATTATGCCCAT
AAAGTTAAATCCCTTATGGCAAGCCTGACGAACATGAGCAGGAGCTCACC
AAGTGAACTCAGTTTAAATGATTCCCTACGTTAG
Human Kv9.2 Protein SEQ Id No.3
MTGQSLWDVSEANVEDGETRINVGGFKRRLRSHTLLRFPETRLGRLLLCH
SREAILELCDDYDDVQREFYFDRNPELFPYVLHFYHTGKLHVMAELCVFS
FSQEIEYWGINEFFIDSCCSYSYHGRKVEPEQEKWDEQSDQESTTSSFDE
ILAFYNDASKFDGQPLGNFRRQLWLALDNPGYSVLSRVFSILSILVVMGS
IITMCLNSLPDFQIPDSQGNPGEDPRFEIVEHFGIAWFTFELVARFAVAP
DFLKFFKNALNLTDLMSIVPFYITLVVNLVVESTPTLANLGRVAQVLRLM
RIFRILKLARHSTGLRSLGATLKYSYKEVGLLLLYLSVGISIFSVVAYTI
EKEENEGLATIPACWWWATVSMTTVGYGDVVPGTTAGKLTASACILAGIL
VVVLPITLIFNKFSHFYRRQKQLESAMRSCDFGDGMKEVPSVNLRDYYAH
KVKSLMASLTNMSRSSPSELSLNDSLR
Mouse Kv9.2 cDNA SEQ ID No.4
ATGACCCGCCAGAGCCTGTGGGATGTGTCCGATACCGACGTCGAGGATGG
AGAGATCCGCATCAATGTGGGTGGCTTCAAGAGACGGCTGCGTTCCCATA
CGCTGCTGCGCTTCCCTGAGACACGCCTGGGCCGTCTGCTCCTCTGCCAC
TCGCGAGAGGCCATTCTGGAACTCTGCGATGACTACGATGACGTTCAGCG
TGAGTTCTACTTCGACCGTAACCCCGAGCTCTTCCCCTATGTGTTGCATT
TCTACCACACCGGCAAGCTTCACGTCATGGCTGAGCTGTGCGTCTTCTCC
TTCAGCCAGGAGATCGAGTACTGGGGTATCAATGAGTTCTTCATCGACTC
TTGCTGCAGCTATAGCTATCACGGCCGCAAAGTGGAACCTGAGCAGGAGA
AATGGGACGAGCAGAGTGACCAGGAAAGCACCACTTCCTCCTTCGATGAG
ATCTTGGCCTTCTATAATGATGCTTCCAAGTTCGATGGGCAACCCCTGGG
CAACTTCCGCAGGCAGCTGTGGCTGGCGTTGGACAACCCAGGCTACTCAG
TCCTAAGCAGGGTCTTCAGTGTCCTTTCCATCTTGGTGGTGTTGGGCTCC
ATCATCACCATGTGCCTCAATAGCCTGCCAGACTTCCAAATCCCTGATAG
CCAGGGTAACCCCGGTGAAGACCCCAGGTTCGAAATTGTGGAGCACTTTG
GCATTGCTTGGTTCACATTTGAGTTGGTGGCCAGGTTTGCTGTGGCCCCT
GACTTTCTTAAGTTCTTCAAGAATGCTCTAAACCTTATTGATCTCATGTC
CATTGTCCCATTTTACATAACTCTAGTGGTGAACCTGGTGGTGGAGAGTT
CTCCTACCTTGGCTAACTTGGGCAGGGTGGCTCAAGTCCTGAGGCTAATG
AGGATCTTCCGAATTCTCAAGCTGGCCAGACACTCCACTGGCCTCCGCTC
CTTGGGAGCCACCCTGAAGTACAGCTACAAGGAAGTGGGGTTGCTCTTGC
TCTACCTCTCAGTGGGGATTTCCATCTTCTCTGTGGTGGCCTACACCATT
GAAAAGGAGGAGAACGAAGGCCTGGCCACCATCCCTGCCTGCTGGTGGTG
GGCCACTGTCAGTATGACCACAGTTGGGTACGGAGATGTGGTCCCAGGGA
CAACAGCTGGGAAGTTGACTGCCTCTGCCTGCATCTTGGCAGGCATCCTG
GTGGTGGTCTTGCCCATCACTTTGATCTTCAATAAGTTCTCCCATTTCTA
TCGGCGCCAAAAGCAACTTGAGAGTGCTATGCGCAGCTGTGACTTTGGAG
ATGGAATGAAAGAGGTCCCTTCGGTCAATTTAAGGGACTACTATGCTCAT
AAAGTTAAGTCCCTCATGGCAAGTCTGACAAACATGAGTAGGAGTTCACC
TAGTGAACTGAGTTTAGATGATTCTCTACATTAG
Mouse Kv9.2 Protein SEQ ID No.5
MTRQSLWDVSDTDVEDGEIRINVGGFKRRLRSHTLLRFPETRLGRLLLCH
SREAILELCDDYDDVQREFYFDRNPELFPYVLHFYHTGKLHVMAELCVFS
FSQEIEYWGINEFFIDSCCSYSYHGRKVEPEQEKWDEQSDQESTTSSFDE
ILAFYNDASKFDGQPLGNFRRQLWLALDNPGYSVLSRVFSVLSILVVLGS
IITMCLNSLPDFQIPDSQGNPGEDPRFEIVEHFGIAWFTFELVARFAVAP
DFLKFFKNALNLIDLMSIVPFYITLVVNLVVESSPTLANLGRVAQVLRLM
RIFRILKLARHSTGLRSLGATLKYSYKEVGLLLLYLSVGISIFSVVAYTI
EKEENEGLATIPACWWWATVSMTTVGYGDVVPGFTAGKLTASACILAGIL
VVVLPITLIFNKFSHFYRRQKQLESAMRSCDFGDGMKEVPSVNLRDYYAH
KVKSLMASLTNMSRSSPSELSLDDSLH

We claim:

1. A method of identifying a compound capable of binding to Kv9.2 polypeptide, the method comprising:
(a) contacting a Kv9.2 polypeptide with a candidate compound; and
(b) determining whether the candidate compound binds to the Kv9.2 polypeptide;
wherein the compound is suitable for treating or alleviating anxiety in an individual, wherein the anxiety is associated with activity of Kv9.2.

2. The method of claim 1, wherein the Kv9.2 polypeptide comprises an amino acid sequence shown in SEQ ID NO: 3 or SEQ ID NO: 5 or a sequence having at least 90% sequence identity thereto.

3. The method of claim 1, wherein the compound is an agonist or an antagonist of Kv9.2 polypeptide.

4. The method of claim 1, wherein the compound is an immunoglobulin.

5. The method of claim 1, wherein the candidate compound is exposed to a cell expressing a Kv9.2 polypeptide.

6. The method of claim 5, wherein a change in conductance of the cell or in kinetics of the Kv9.2 polypeptide is detected.

7. The method of claim 6, wherein an increase in conductance or kinetics is detected, thereby identifying an agonist of Kv9.2.

8. The method of claim 6, wherein a decrease in conductance or kinetics is detected, thereby identifying an antagonist of Kv9.2.

9. The method of claim 1, wherein the anxiety is selected from the group consisting of social anxiety, post traumatic stress disorder, phobias, social phobia, special phobias, panic disorder, obsessive compulsive disorder, acute stress disorder, separation anxiety disorder, generalised anxiety disorder, major depression, dysthymia, bipolar disorder, seasonal affective disorder, post natal depression, manic depression, bipolar depression, anxiety, anxiety disorders, anxiety-related behaviour, generalized anxiety disorder, agoraphobia, acute stress disorder, panic disorders, and depression.

10. The method of claim 1, further comprising:
(c) administering the compound capable of binding to Kv9.2 polypeptide to an animal that does not express functional Kv9.2 polypeptide; and
(d) determining whether the compound produces a physiological response in the animal.

11. The method of claim 10, wherein the physiological response is selected from the group consisting of changes to disease resistance; altered inflammatory response; altered tumour susceptibility; a change in blood pressure; neovascularization; a change in eating behavior; a change in body weight; a change in bone density; a change in body temperature; a change in insulin secretion; a change in gonadotropin secretion; a change in nasal and/or bronchial secretion; vasoconstriction; loss of memory; anxiety; hyporeflexia; hyperreflexia; and changes in pain or stress responses, compared with an animal that does not express functional Kv9.2 polypeptide to which the compound is not administered.

12. A method of identifying a compound for treating or alleviating anxiety comprising:
(a) administering a candidate compound capable of binding to Kv9.2 polypeptide to an animal; and
(b) determining whether the animal exhibits a change in anxiety;
thereby identifying a compound for treating or alleviating anxiety.

13. The method of claim 12, wherein the Kv9.2 polypeptide comprises an amino acid sequence shown in SEQ ID NO: 3 or SEQ ID NO: 5 or a sequence having at least 90% sequence identity thereto.

14. The method of claim 12, wherein the animal expresses functional Kv9.2 polypeptide.

15. The method of claim 12, wherein the animal is a wild type animal.

16. The method of claim 12, wherein the animal is a rodent.

17. The method of claim 12, wherein the animal is a mouse.

18. The method of claim 16, wherein anxiety is measured using an Open Field Test or a Plus Maze Test.

19. The method of claim 12, wherein the anxiety is selected from the group consisting of social anxiety, post traumatic stress disorder, phobias, social phobia, special phobias, panic disorder, obsessive compulsive disorder, acute stress disorder, separation anxiety disorder, generalised anxiety disorder, major depression, dysthymia, bipolar disorder, seasonal affective disorder, post natal depression, manic depression, bipolar depression, anxiety, anxiety disorders, anxiety-related behaviour, generalized anxiety disorder, agoraphobia, acute stress disorder, panic disorders, and depression.

20. The method of claim 12, further comprising:
(c) administering the compound capable of binding to Kv9.2 polypeptide to an animal that does not express functional Kv9.2 polypeptide; and
(d) determining whether the compound produces a physiological response in the animal.

21. The method of claim 20, wherein the physiological response is selected from the group consisting of changes to disease resistance; altered inflammatory response; altered tumour susceptibility; a change in blood pressure; neovascularization; a change in eating behavior; a change in body weight; a change in bone density; a change in body temperature; a change in insulin secretion; a change in gonadotropin secretion; a change in nasal and/or bronchial secretion; vasoconstriction; loss of memory; anxiety; hyporeflexia; hyperreflexia; and changes in pain or stress responses, compared with an animal that does not express functional Kv9.2 polypeptide to which the compound is not administered.

22. A method of identifying an agonist of Kv9.2 polypeptide, the method comprising:
(a) administering a candidate compound capable of binding to Kv9.2 polypeptide to an animal; and
(b) determining whether the animal exhibits an increase in anxiety;
thereby identifying an agonist of Kv9.2 polypeptide.

23. A method of identifying an antagonist of Kv9.2 polypeptide the method comprising:
(a) administering a candidate compound capable of binding to Kv9.2 polypeptide to an animal; and
(b) determining whether the animal exhibits a decrease in anxiety;
thereby identifying an antagonist of Kv9.2 polypeptide.

24. A method of treating an individual suffering from anxiety, wherein the anxiety is associated with Kv9.2 activity, the method comprising administering an antagonist of Kv9.2 to the individual.

25. A method of diagnosing susceptibility to anxiety in an individual, wherein the anxiety is associated with Kv9.2 activity, the method comprising detecting a change in expression pattern or level of Kv9.2 in a cell or tissue of the individual.

26. A method of diagnosing susceptibility to anxiety in an individual, wherein the anxiety is associated with Kv9.2 activity, the method comprising detecting a polymorphism in a Kv9.2 polynucleotide in a cell or tissue of the individual.

27. The method of claim 25, wherein the Kv9.2 polynucleotide comprises a nucleic acid sequence shown in SEQ ID NO: 1, SEQ ID NO: 2, or SEQ ID NO: 4.

28. A transgenic non-human mammal comprising a disruption in the endogenous Kv9.2 gene, wherein the disruption results in a change in anxiety in the mammal.

29. A cell or tissue isolated from the transgenic non-human mammal of claim 28.


SEQUENCE LISTING (21 sequences)

SEQ ID NO: 1 - DNA, 5195 bp, Homo sapiens
gcctctctgggtgggtgaggggcgcgcggatccggagagggggctccgggagcggcgggaccacgcagccacctgtgagccttcggcagctgcgggcggcggcggcgtacccggcccgagacgggaggagacgctcggcggcccccgcccgccggcccgccgggcgcacacactcgcacccgcgcacgcaccgccagcaggcagcggccaccgccgcgatgctcgcccgcgggttggggaagtttcccgccggcctcggccgcgggcacccgtgctcccaggtgtagcgcccccgcgcggcgcgggcggccggcgcctccagcatgaccggccagagcctgtgggacgtgtcggaggctaacgtcgaggacggggagatccgcatcaatgtgggcggcttcaagaggaggctgcgctcgcacacgctgctgcgcttccccgagacgcgcctgggccgcttgctgctctgccactcgcgcgaggccattctggagctctgcgatgactacgacgacgtccagcgggagttctacttcgaccgcaaccctgagctcttcccctacgtgctgcatttctatcacaccggcaagcttcacgtcatggctgagctatgtgtcttctccttcagccaggagatcgagtactggggcatcaacgagttcttcattgactcctgctgcagctacagctaccatggccgcaaagtagagcccgagcaggagaagtgggacgagcagagtgaccaggagagcaccacgtcttccttcgatgagatccttgccttctacaacgacgcctccaagttcgatgggcagcccctcggcaacttccgcaggcagctgtggctggcgctggacaaccccggctactcagtgctgagcagggtcttcagcatcctgtccatcctggtggtgatggggtccatcatcaccatgtgcctcaatagcctgcccgatttccaaatccctgacagccagggcaaccctggcgaggaccctaggttcgaaatcgtggagcactttggcattgcctggttcacatttgagctggtggccaggtttgctgtggcccctgacttcctcaagttcttcaagaatgccctaaaccttattgacctcatgtccatcgtccccttttacatcactctggtggtgaacctggtggtggagagcacacctactttagccaacttgggcagggtggcccaggtcctgaggctgatgcggatcttccgcatcttaaagctggccaggcactccactggcctccgctccctgggggccactttgaaatacagctacaaagaagtagggctgctcttgctctacctctccgtggggatttccatcttctccgtggtggcctacaccattgaaaaggaggagaacgagggcctggccaccatccctgcctgctggtggtgggctaccgtcagtatgaccacagtggggtacggggatgtggtcccagggaccacggcaggaaagctgactgcctctgcctgcatcttggcaggcatcctcgtggtggtcctgcccatcaccttgatcttcaataagttctcccacttttaccggcgccaaaagcaacttgagagtgccatgcgcagctgtgactttggagatggaatgaaggaggtcccttcggtcaatttaagggactattatgcccataaagttaaatcccttatggcaagcctgacgaacatgagcaggagctcaccaagtgaactcagtttaaatgattccctacgttagccgggaggacttgtcaccctccaccccacattgctgagctgcctcttgtgcctctggcacagcccaggcaccttatggttatggtgtaaggagtatgcccagcccctgaggggagagatgcatgggatatgcacccaggtttcttttacagtttttagaatcgtttttagagggtggtgtgtctgacaccatgcctttgcacctttccatgaaatgacactcactggtctttgcatcgtgggcataaaatgttcacctttttgccagatgagtacacccagaatgctaatttttctgtccatcgtgtacgctattctagtgcttgtggcccagtactgtctatgagttgtcgtgctcctgtttctgaggttgtcgtgtgagttctgtacaaaaagcccccacaagtcgtccagtagaaatgcatctatgaggtcagcaaggatatgatgagattttgctcacagtcatgtgaaaacaaaatctcagctctttatccattgctttcacttagttttagtaccaaaacaaagagaatgcaaagttaagcagacttgaccaatgcaagtctctaagttgtttttataaatgatctgtagttccgtggcttgcatgggtgcaccaatcatctttagaacgatgtacactgatgttcatctcataaatgtcactctttagagaatgttacttagttaaacatgcagtgaagatcgaatttttttcccaagaacagatgtgttagggagaggggcttcagctaaatagtccaaaccctagggtgcttaaagccaagttagtgcaggctgagccccttggttcacagtcaagcctccttgtttcctagggtgactgtagagaaatgtatttccggatgaggtttctgatctaggccatttgaccaaactttgctgtgtctaagatattagcatgtttttgaaatatttattttttaagatgtttaggagtaaggtcgtgttgtcttcctcaactaaaaagaagtttactgttgtatcgtctccctgaggtgaacgttgttgggttgctagcaagggcagtagcttaaatacttttgttgcctactctgaaagctcatcaaatgagagcccttttatttccaagcagaatttagtcagataattttgcttctaggatatagtatgttgtatatgatgctgtgattgccctggagttcctgccatgacatggaaacctggtggtatggaagcatgtactcaaaatatagacgtgcacgatggtggtgtggcttacccaggatggaaacactgcagttcttacttgcattcccactgcctttcatggggggtgactgggtagaggccaggagaaaggaaagagttgtaaaataaaaaactgctagttcataaaatgtcataaaaaattgtaaacttgaaaagcttaatgctattcaaaagaccttcaagcttccaaacttgtattgaagggagacgactgtttcctcctccaaaatgctcctgctcctcttgttcggttaaccagcacataacattgtgatggggaacctgggttcctctataagataattcttctccatcatctttaaggtaatctgatggttttccaggtggctttcattattgttccatctttgaaaaggcaatagaacccaggggtctgagcatggagctatccagggttttcatccaaaggttgggcctcttcttaagaggtccttttgtgtttcagttgattgaagatgatacttacctcattggaggtgtggcaaggatcttatcagaaggctttgtgttcttgtagttgtcatggctactacagtgtgggtgatttattgaatgaattcactagccacttgtgtcctggagcccccagttcaaatctttccattggactggaggcttgtgggaggctgggaggtggctgtctcctagtgtctacatccgtgtctctgaagcatcaggaaaagtgagatgacttagaggcaactgggcactgaatcagaggagcagagttatttttcagaatttgcacatggaacacttagatttggctggtgcttccagccctggaaggcataacatttacggactcatccccagctgcactgaaggcaggtggtggtacagacttatgaggacggatcagtttgccaaggctgatggtattgggtcactgagcctggtatccatggccgctgaccaggaagcttatgcaaagtggaagcaaggaacaaggcagaataactcagtcactttcatgaagatttttctaaacaagaaggcttaccaccaaaaaagaggtaccctagtggttaccctttgcagatgtgaaagctggaaaacttgacttttctttttggtaatgacttgcatttatctggtgcctttcgttggaggaatcccaacgtgctttagagactatctttttaacatctcttgtacatacatatatacttatataaaatattatcttgcccaactggacctttactcacttctgagcatgagaatgtcccaatagcattgagtttttcaagtggtggtttcagataagtgggagaaagaacaacccggctggcttaaaccctggagctaattcccacaaggaatgtagactgaatggtgacccagggagaaataatcttcctctcccctaaagtctcactaaggtttgaagtttacaggtgctctccactgggtctttgatcgaccttgctagataacatctaactaaaagcagtttcttttagtccctgaagctaaccagggagagtcaggttaattttctgtaaaaatatgaggtgacatctttggcaaccaggctgtcagactgacctgtaaacctcctttagggggacagagtagaaactggagatgacttgtttccagctgtgagcttgagagaagtgtcactcccagcatttgaaggttattgttttcaatgccagtgggccaaatatatgggccaggctttgatatctgtgatgtgcattttggaagtgctgggttgggaagtgacacgtctgttgcacaaatgcatattggttataggtttgtgttttctgccaaacccccacatttctcgggtttgtgagtgaggaagggcatgttgtaatgccaagctgatttgtagctcgtaaggtagtaattggtatttaacatttgcatttgttatttctacttatcttagcactcaaataattgaactacctgctaattcttgccgcatttcaaagaaaataagttgttatgcactttgggatagtggtgatctgtacaggctgtgtgttagctacttgaaggcgtaactggtatttcttgtgtgttttaacagcatgacttcttacagagctgtaatttttaaaattgaggatgccatatttgagatgtcagttttaacactcattaacacactactgtgcaagcattgacacaggctgcactg

SEQ ID NO: 2 - DNA, 1434 bp, Homo sapiens
atgaccggccagagcctgtgggacgtgtcggaggctaacgtcgaggacggggagatccgcatcaatgtgggcggcttcaagaggaggctgcgctcgcacacgctgctgcgcttccccgagacgcgcctgggccgcttgctgctctgccactcgcgcgaggccattctggagctctgcgatgactacgacgacgtccagcgggagttctacttcgaccgcaaccctgagctcttcccctacgtgctgcatttctatcacaccggcaagcttcacgtcatggctgagctatgtgtcttctccttcagccaggagatcgagtactggggcatcaacgagttcttcattgactcctgctgcagctacagctaccatggccgcaaagtagagcccgagcaggagaagtgggacgagcagagtgaccaggagagcaccacgtcttccttcgatgagatccttgccttctacaacgacgcctccaagttcgatgggcagcccctcggcaacttccgcaggcagctgtggctggcgctggacaaccccggctactcagtgctgagcagggtcttcagcatcctgtccatcctggtggtgatggggtccatcatcaccatgtgcctcaatagcctgcccgatttccaaatccctgacagccagggcaaccctggcgaggaccctaggttcgaaatcgtggagcactttggcattgcctggttcacatttgagctggtggccaggtttgctgtggcccctgacttcctcaagttcttcaagaatgccctaaaccttattgacctcatgtccatcgtccccttttacatcactctggtggtgaacctggtggtggagagcacacctactttagccaacttgggcagggtggcccaggtcctgaggctgatgcggatcttccgcatcttaaagctggccaggcactccactggcctccgctccctgggggccactttgaaatacagctacaaagaagtagggctgctcttgctctacctctccgtggggatttccatcttctccgtggtggcctacaccattgaaaaggaggagaacgagggcctggccaccatccctgcctgctggtggtgggctaccgtcagtatgaccacagtggggtacggggatgtggtcccagggaccacggcaggaaagctgactgcctctgcctgcatcttggcaggcatcctcgtggtggtcctgcccatcaccttgatcttcaataagttctcccacttttaccggcgccaaaagcaacttgagagtgccatgcgcagctgtgactttggagatggaatgaaggaggtcccttcggtcaatttaagggactattatgcccataaagttaaatcccttatggcaagcctgacgaacatgagcaggagctcaccaagtgaactcagtttaaatgattccctacgttag

SEQ ID NO: 3 - protein, 477 aa, Homo sapiens
MTGQSLWDVSEANVEDGEIRINVGGFKRRLRSHTLLRFPETRLGRLLLCHSREAILELCDDYDDVQREFYFDRNPELFPYVLHFYHTGKLHVMAELCVFSFSQEIEYWGINEFFIDSCCSYSYHGRKVEPEQEKWDEQSDQESTTSSFDEILAFYNDASKFDGQPLGNFRRQLWLALDNPGYSVLSRVFSILSILVVMGSIITMCLNSLPDFQIPDSQGNPGEDPRFEIVEHFGIAWFTFELVARFAVAPDFLKFFKNALNLIDLMSIVPFYITLVVNLVVESTPTLANLGRVAQVLRLMRIFRILKLARHSTGLRSLGATLKYSYKEVGLLLLYLSVGISIFSVVAYTIEKEENEGLATIPACWWWATVSMTTVGYGDVVPGTTAGKLTASACILAGILVVVLPITLIFNKFSHFYRRQKQLESAMRSCDFGDGMKEVPSVNLRDYYAHKVKSLMASLTNMSRSSPSELSLNDSLR

SEQ ID NO: 4 - DNA, 1434 bp, Mus musculus
atgacccgccagagcctgtgggatgtgtccgataccgacgtcgaggatggagagatccgcatcaatgtgggtggcttcaagagacggctgcgttcccatacgctgctgcgcttccctgagacacgcctgggccgtctgctcctctgccactcgcgagaggccattctggaactctgcgatgactacgatgacgttcagcgtgagttctacttcgaccgtaaccccgagctcttcccctatgtgttgcatttctaccacaccggcaagcttcacgtcatggctgagctgtgcgtcttctccttcagccaggagatcgagtactggggtatcaatgagttcttcatcgactcttgctgcagctatagctatcacggccgcaaagtggaacctgagcaggagaaatgggacgagcagagtgaccaggaaagcaccacttcctccttcgatgagatcttggccttctataatgatgcttccaagttcgatgggcaacccctgggcaacttccgcaggcagctgtggctggcgttggacaacccaggctactcagtcctaagcagggtcttcagtgtcctttccatcttggtggtgttgggctccatcatcaccatgtgcctcaatagcctgccagacttccaaatccctgatagccagggtaaccccggtgaagaccccaggttcgaaattgtggagcactttggcattgcttggttcacatttgagttggtggccaggtttgctgtggcccctgactttcttaagttcttcaagaatgctctaaaccttattgatctcatgtccattgtcccattttacataactctagtggtgaacctggtggtggagagttctcctaccttggctaacttgggcagggtggctcaagtcctgaggctaatgaggatcttccgaattctcaagctggccagacactccactggcctccgctccttgggagccaccctgaagtacagctacaaggaagtggggttgctcttgctctacctctcagtggggatttccatcttctctgtggtggcctacaccattgaaaaggaggagaacgaaggcctggccaccatccctgcctgctggtggtgggccactgtcagtatgaccacagttgggtacggagatgtggtcccagggacaacagctgggaagttgactgcctctgcctgcatcttggcaggcatcctggtggtggtcttgcccatcactttgatcttcaataagttctcccatttctatcggcgccaaaagcaacttgagagtgctatgcgcagctgtgactttggagatggaatgaaagaggtcccttcggtcaatttaagggactactatgctcataaagttaagtccctcatggcaagtctgacaaacatgagtaggagttcacctagtgaactgagtttagatgattctctacattag

SEQ ID NO: 5 - protein, 477 aa, Mus musculus
MTRQSLWDVSDTDVEDGEIRINVGGFKRRLRSHTLLRFPETRLGRLLLCHSREAILELCDDYDDVQREFYFDRNPELFPYVLHFYHTGKLHVMAELCVFSFSQEIEYWGINEFFIDSCCSYSYHGRKVEPEQEKWDEQSDQESTTSSFDEILAFYNDASKFDGQPLGNFRRQLWLALDNPGYSVLSRVFSVLSILVVLGSIITMCLNSLPDFQIPDSQGNPGEDPRFEIVEHFGIAWFTFELVARFAVAPDFLKFFKNALNLIDLMSIVPFYITLVVNLVVESSPTLANLGRVAQVLRLMRIFRILKLARHSTGLRSLGATLKYSYKEVGLLLLYLSVGISIFSVVAYTIEKEENEGLATIPACWWWATVSMTTVGYGDVVPGTTAGKLTASACILAGILVVVLPITLIFNKFSHFYRRQKQLESAMRSCDFGDGMKEVPSVNLRDYYAHKVKSLMASLTNMSRSSPSELSLDDSLH

SEQ ID NO: 6 - DNA, 27 bp, Artificial Sequence
  Description of Artificial Sequence Synthetic primer
ctctcaattcaggtggcacccttagag

SEQ ID NO: 7 - DNA, 27 bp, Artificial Sequence
  Description of Artificial Sequence Synthetic primer
cacagaattcccaatcataagacatag

SEQ ID NO: 8 - DNA, 27 bp, Artificial Sequence
  Description of Artificial Sequence Synthetic primer
ctctcaattcaggtggcacccttagag

SEQ ID NO: 9 - DNA, 36 bp, Artificial Sequence
  Description of Artificial Sequence Synthetic primer
aaaaccggtatgtccagatcctcatacatggcacac

SEQ ID NO: 10 - DNA, 38 bp, Artificial Sequence
  Description of Artificial Sequence Synthetic primer
aaagcggccgcgacgtcggtatcggacacatcccacag

SEQ ID NO: 11 - DNA, 38 bp, Artificial Sequence
  Description of Artificial Sequence Synthetic primer
tttggcgcgccttgctgatctgctgcttgtggttctag

SEQ ID NO: 12 - DNA, 38 bp, Artificial Sequence
  Description of Artificial Sequence Synthetic primer
aaaggccggccaatgtaaccatcgcttctgtaacccag

SEQ ID NO: 13 - DNA, 27 bp, Artificial Sequence
  Description of Artificial Sequence Synthetic primer
agcagagcaggtatggcgtggcatgtc

SEQ ID NO: 14 - DNA, 27 bp, Artificial Sequence
  Description of Artificial Sequence Synthetic primer
ctgggggagctctcgtgctatgatgag

SEQ ID NO: 15 - DNA, 27 bp, Artificial Sequence
  Description of Artificial Sequence Synthetic primer
cccatttctatcggcgccaaaagcaac

SEQ ID NO: 16 - DNA, 27 bp, Artificial Sequence
  Description of Artificial Sequence Synthetic primer
gtgctagaaccacaagcagcagatcag

SEQ ID NO: 17 - DNA, 27 bp, Artificial Sequence
  Description of Artificial Sequence Synthetic primer
cagccgaactgttcgccaggctcaagg

SEQ ID NO: 18 - DNA, 27 bp, Artificial Sequence
  Description of Artificial Sequence Synthetic primer
catgccgcctgcgccctattgatcatg

SEQ ID NO: 19 - DNA, 8000 bp, Artificial Sequence
  Description of Artificial Sequence Synthetic construct
  CDS (2063)..(3493)
aatctagagccagaacacctctcaattcaggtggcacccttagagccacatatgaagaatgtgtttctcgtggttgcactgtttttcctgagtctggagccaccttataagaactgtcctattacttctgaccaagacatgaggacaccagctctctgctaacaatgcatgccacagtctgaaaatgaggaccatgtatttgctcagagaccagggggataccaaggaataagggtctatttcttggcttggccaatatgagggccctatgtcttatgattgggaattctgtgctgaagtcagtgtttatacagcacacagtttctcacactgtatgcacataccacactaatgcatgtccagatcctcatacatggcacactgtatgcatcttcgcatccatcagaagacatatttgtcaatactttacacagattttgggtagtcctactcctcagattgcacacacgcacagaactccaagtactcctctgaagctcacacactacactggcttgtacatacacggtatcatgcagagttctcaaacagaactccattactcctccccatgccaacaggacctgtgcacacacccaagctcctgccccacccatatccctgccacagctcaacctcagtcttctctgacagctcccattttccgataaccccatttccaggtataggaaacttttcttcaggtttctaaaagaggaagccgaagccggtagatcattccgttgctgccctatccgccctatacataaaagccatccttcattctccagtctgtttcctacagacccggcgagggaggaaagtcactgtaagcaaccctctgtctgagccctgggggtgctgacaataacagctgtccctggaggatgccgagggagggggaaaaggtgtcagctccgtgcaaagctggggggcgcccgaagaacagaatgatgctccggaacgtctcaagagtctgggcggcgactgtgccccgggctggcgcgctcggagctgtccgttcagcaccaccgcgcagcaccaggctcaaggccctctgcaaggcgcaccggctcggttcccgcccccgctccacgggcgcctgcggcgtgggctcctgcctctcgtggtgggtgaggggcgcgcagatcggaagaggggggctccacgagcgtcggggccacgcagccacctgtgagcctctcgcggatgtgggcggtggtgtgtgcggccggagactgaaggaggcgcacggtggaccccgcctgcccggtggcggggcacacagacaagcactcacatctacatcctcgcacccgcgcgcactcgcgcagccagccaacggcgtccaccgcggtgatgcttgccagcaggtcggggaagtttcccgccggcctctgccgcgggctcccgtgcacccaggtaagcgcttttagaacgcgcaaggcgagctccgggaaggcgcagcgcacgcggccggggagcacggcgccagaagggcgcgggggtggtggtaggaagggggctgggaatggaatcggtccttgaaggctggagatctgggacgctgagttgacccttttagccctcggcccagatttacagattagagcggtgaatttccttgcctcctcccaaatctccgcgcccccttcttggcccagccctgccagtgcgcatctcagcttgggtcccgcctgtctgcggcgagagggcgggggcgtctccttggtctgctcacaggcaaggtcagcacacagcccccttgggctttcagagccgacaggcgccactccctggaggaggtggaggcccgggtgtactcgtggaaacatacacccttgtcttgccttgggaagggagtcaactcccatagacccattcctgcaccccagtgctggacctcacctagagaccctgctggagaggccagtcagatgagggtgaagagaaaacaagagaaagacttggggtggggagtgccggcgctcacatagatgctgttccctctgctttcaggtgtagcgcccccgcgcggcgcgggcgcctgggcatctccagcatgacccgccagagcctgtgggatgtgtccgataccgacgtcgaggatggagagatccgcatcaatgtgggtggcttcaagagacggctgcgttcccatacgctgctgcgcttccctgagacacgcctgggccgtctgctcctctgccactcgcgagaggccattctggaactctgcgatgactacgatgacgttcagcgtgagttctacttcgaccgtaaccccgagctcttcccctatgtgttgcatttctaccacaccggcaagcttcacgtcatggctgagctgtgcgtcttctccttcagccaggagatcgagtactggggtatcaatgagttcttcatcgactcttgctgcagctatagctatcacggccgcaaagtggaacctgagcaggagaaatgggacgagcagagtgaccaggaaagcaccacttcctccttcgatgagatcttggccttctataatgatgcttccaagttcgatgggcaacccctgggcaacttccgcaggcagctgtggctggcgttggacaacccaggctactcagtcctaagcagggtcttcagtgtcctttccatcttggtggtgttgggctccatcatcaccatgtgcctcaatagcctgccagacttccaaatccctgatagccagggtaaccccggtgaagaccccaggttcgaaattgtggagcactttggcattgcttggttcacatttgagttggtggccaggtttgctgtggcccctgactttcttaagttcttcaagaatgctctaaaccttattgatctcatgtccattgtcccattttacataactctagtggtgaacctggtggtggagagttctcctaccttggctaacttgggcagggtggctcaagtcctgaggctaatgaggatcttccgaattctcaagctggccagacactccactggcctccgctccttgggagccaccctgaagtacagctacaaggaagtggggttgctcttgctctacctctcagtggggatttccatcttctctgtggtggcctacaccattgaaaaggaggagaacgaaggcctggccaccatccctgcctgctggtggtgggccactgtcagtatgaccacagttgggtacggagatgtggtcccagggacaacagctgggaagttgactgcctctgcctgcatcttggcaggcatcctggtggtggtcttgcccatcactttgatcttcaataagttctcccatttctatcggcgccaaaagcaacttgagagtgctatgcgcagctgtgactttggagatggaatgaaagaggtcccttcggtcaatttaagggactactatgctcataaagttaagtccctcatggcaagtctgacaaacatgagtaggagttcacctagtgaactgagtttagatgattctctacattagctggaccccgacttacattgctgatctgctgcttgtggttctagcacaatcagggcaattttagggctgtggcataagaaatcatccctgccctagagggagagctgcatgggacataagccctagattgcttttgcaatgtttagagaggtttcttttttcttttgaggatggtgtgtctaataacatgcctttgcacctctcagtgaagtgacactcactggtgtttgcatcatggcaaaaaaaaaaatgttcacctttctgccagatgagtatctagaatgccaatttctctgtccactgtgtacagtattctaatgctcatatcccagcattacctgtgagtggaattgtctgtgctcctatttccgaggctgctgtatgggttccagtgacaacacatctgtctatgaggtcagcaaggatatcgtgagatttggatcacaaccatgtgaaaataatctcaattatgtatcccttgctttcatttacctttaataccaaaacagagagatcgcaaagctaagcacacttgaccaatgcaaatcttcgagttgtctttctacttggtcctgtccttgtgatgtgcatgaatgcaccagtcattttaaagaaagatatgtattgatgtatatctcctaagtgtcagtgtaaagagaatgttacttagtcgatatgtagtaaagactgaatgtttttcctccaaacagttaaatttagggacagcgactttagctaaacatggaccaaacccccaggagttcatgtaggctaagcccttttactgatggtcaggcctcttttcatttatttctggctgagatgctcaatccaggccattttgaccaaagtttgctgtgtcttggtattagcatgtttttcaagcatctcttttttaagatgtttaggaataaggccgtgctgtctttctcctccactggaagaagtttgtgttttgttgtctttgtgaggtaagcactgtcaggttgctggcaagggcaatagcttaaatattttcttgcctgctctaaaagcccataaaatgaatgttcttttgtttctgagcagagtttcctttaggtagttttgcttctaggacacaggatagtgtatgtatagtgtttgattgccttgagttcctgccttggcatggaaacctggtagtgcagagcatattcaagatgcagacatgcatgaaggcaggtggcttacccagggtggaaacactgtagcttttatttccattgcaattgcaatggggtttgggggacaggggtagaagtcaaaagaaaagagttataaagccaaaaactactttataagatataaataactgtgagcttctttaaaagcttaaaactagtaaaatagaaaacaaaaacaacaaacaaccctttcaaccattaagctgtgttgaagggttacctctatttccttttccaacacactgttcagttaacacaaaacattatgaaggggcacctgggccccctttaagaaaattctttcccatcattgtctaagtaatctgatggttttccagtggctttcattattgttgagtctttggcaaggctatagaatccagggatccaaatacggagataaacaggcttttaatccaaagtttgggcctactcttaattggtccattttgtgttttagtcagttaaggacaatccacacttcacaggggtgtggaatggatcttctcagagggctctgtgtactagtggttgccatggtgactacagtgtgggcgatttgttggatgaatgcactgaccatttaatgcccttgagcctccagttcagatctttcatcagactggagactgatgaggggcagaaggtggcaacctcctagtgcctacgtgcagacacatctatgtctattattccaaagcagcaggaatgtgaggttgacttcaaggaaccccgcctgtgaatcagaagacaggagttggttttgggagtttgcatatggaactcttgttgttggctggtatcttcagcactagaagaccaaggatgtataaaactgttcccagttgcactgaaggcaggggacagcctaacctgtgaggactgtttggtttgctgaggctgatggtataccataactgggcatgaattgcatggtcactgaccatgaagctgatggaaagaagaaaagaggagatgcagagtaacttagccactctcctaaggatttttctaaatgaacatgtttaacaccaaaaaggagatacctgcaaggatatgaaagctggaaaacttgacttttcttttttggtgatgacttgttttatctggtgcctttcattgggggaatcccaaagtgctttagagactgtctttttaacatttcttgtagatatatgtataattgtaaaagaatattcctttgcccaccaagacttttagtcgcttctgagcatgaaaaggtcccaggagcattaagttcccccaagcagtagtttcatagaccttgagggagggccatccagatggctgggctctgcagtgatctccatagaccataaagaatggtgtaaaggccctgggaacctttcccttatcactgaactacacggaggtatgaagttcacaagtcctggatcagacacaaagcctctgactagtcaagtcagataatgtctcccatggtagtttctcctccaggagagagcagattcattttctgccgtgttcttgggagtaaaggctaccaagatgaagtggactcatagatgatcaaggtagcctgtacaatcctttcttggggaacacagtagatgcaggctcattcatacccagctggtagctaaagagcaactccactcttggcatttgacctgttcagtattactgggtcaaatacatgggccaagctttgtcatagcatgcagggtgggcatttggaactgacacacgtttatggcacagatacattagtctggggctgtgtttcccatccaagttctgcatttcccaggttggctgctgaggaggggcgtgtagaatgctgagcctatgcctagctcatcaggtagtaattgatgtttaatatttgggtttgttattgctacttactgtagctctcaaagcactgagcgacctgttaattcctgctttgtttcaatggaaatgatttgttctgcactttgggatatcggcggtagagaccacaggcagtgtggtctctacttgaaagcttaactggtatttcttgtatgttttaacagcatgacttgttccagggttgtaattttaaacatcgagaatactgtatttgcgatgtcagttttaacactcattaacacactactgtgccagcgtcctggcggctcctgcgccattacatcgctgctgtgcttgagtttcttgtgcctcgactccagaatgaggcacatgactgacatactcaggatgccagccttgcaaatatgcagattaaacagaagataattatttcacttccctaaggtccctcaattacacatggagtggcagagataaggacattgggaagcaagggattggactgcccaagaaaggctgaactggtgttttgctttgttttgttttgttttgttttgtttgtttttgtttttggggattttttgttttgttttgttttttggttgtttatggtgtgtgtgtgtgtgtgtgtgtgtgtgtgtgtgtgtgtgtgtgcatagttacacatgtgtagaggttagggttaatgttggatattttcctcaactactctgcaccttatatattcaggagtgatctctcatttgaacccagaccccagcggtcttgctagtctagcttgccagcttgctttgggaatcattgcttctgcctccatgggctgcgattataggtagacactcatcctgcctcccatcctgggtttggggatctaaatgatggtcttctcagccccaaggctgagcagtgagtgagaagggaaatttccttcttagcaggcagctgaggaaggagcctctgctgagatctggagctactgggttacagaagcgatggttacattgtcttgggaccccaggggactggaggttcctataagattttcttgctgctcagtgtcccacattgagcctccatagcctgctctgaccaccctgttctgtcccataggaccaatccttttgcaactcaagtggttagtgatagcagagcaggtatggcgtggcatgtccaggctggttggctgtgaacattgttagaggatccctgaacttggctccttgctctcccttgctcgtccactgctgcagagtgaggaattggatggaataattcataaagccctgtccacttgtttaccttggtatgaaagcagaatttctgtgtgcctctccatgtcctcatcatagcacgagagctcccccagcccctgattgattttaaggaaggtagaaggactgtttacatacaaggtcagacagggacctggagaaaggcttgggcctactgtctgcttcaag

SEQ ID NO: 20 - protein, 477 aa, Artificial Sequence; identical to SEQ ID NO: 5
  Description of Artificial Sequence Synthetic construct

SEQ ID NO: 21 - protein, 6 aa, Artificial Sequence
  Description of Artificial Sequence Synthetic 6xHis tag
HHHHHH